(19) United States
(12) Patent Application Publication
(10) Pub. No.: US 2011/0190399 A1
(43) Pub. Date: Aug. 4, 2011

(54) CURCUMIN NANOPARTICLES AND METHODS OF PRODUCING THE SAME

(75) Inventors: Santosh Kumar Kar, New Delhi (IN); Feroz Akhtar, New Delhi (IN); Gopesh Ray, New Delhi (IN); Atul Kumar Pandey, New Delhi (IN)

(73) Assignee: Santosh Kumar Kar, New Delhi (IN)

(21) Appl. No.: 13/056,515

(22) PCT Filed: Jul. 31, 2009
(86) PCT No.: PCT/IB2009/053342

§ 371 Date: Apr. 21, 2011

(30) Foreign Application Priority Data
Jul. 31, 2008 (IN) 1827/DEL/ 2008

(51) Int. Cl. A61K 31/12; C07C 49/255; C08B 37/08; B32B 1/00; A61P 35/00; A61P 29/00; A61P 25/28; A61P 3/10; A61P 1/16; A61P 31/00; A61P 33/00; A61P 33/06; A61P 25/00; B82Y 5/00

(52) U.S. Cl. 514/679; 568/325; 428/402; 536/20; 428/403; 977/773

(57) ABSTRACT

The present invention provides for curcumin nanoparticles and curcumin bound to chitosan nanoparticles and methods of producing the same. Bioavailability of curcumin in these formulations was shown to improve by more than 10 fold.

### FIELD OF INVENTION

The present invention deals with curcumin nanoparticles and curcumin bound to chitosan nanoparticles which enhance curcumin bioavailability.

### BACKGROUND OF THE INVENTION

Curcumin a polyphenolic component of the plant Curcuma longa is an interesting molecule because of the variety of biological activities it possesses. Prominent among them are anti-inflammatory and cancer chemopreventive activities (Ammon et al. Pharmacology of Curcuma longa, Planta Med., 1-7, 1991). Curcumin's effect on proteins whose abnormal functioning leads to Alzheimer's disease demonstrates the possibility of developing better drugs for the same disease using curcumin or its derivatives. (Ringman et al. A Potential Role of the Curry Spice Curcumin in Alzheimer's Disease. Curr Alzheimer Res 2005; 2:131-136).

Curcumin has been shown to possess wide range of pharmacological activities including antimicrobial effect (Negi et al., 1999. Antibacterial Activity of Turmeric Oil: A Byproduct of curcumin Manufacture, Journal of Agricultural and Food Chemistry 47(10), 4297-4300), reducing the incidence of cholesterol gallstones (Hussain et al., 1992 Effect of curcumin on cholesterol gall- stone induction in mice, Indian J. Med. Res., 96: 288-291), protection of liver injury from both alcohol and drugs (Nanji et al. 2003 Curcumin prevents alcohol-induced liver disease in rats by inhibiting the expression of NF-kappa B-dependent genes, Am. J. Physiol. Gastrointest. Liver Physiol., 284 (2), G321-327, and Venkatesan et al., 1995, G., Modulation of cyclophosphamide-induced early lung injury by curcumin, an anti-inflammatory antioxidant, Mol. Cell. Biochem., 142 (1), 79-87). Recently its in vitro anti-parasitic activity against Leishimania has been described (Saleheen et al., 2002. Latent activity of curcumin against leismaniasis in vitro. Biol. Pharm. Bull. 25, 386-389.) and it has the ability to hinder Trypanosoma and Plamodium viability (Nose et at., 1998 Trypanocidal effects of curcumin in vitro, Biol. Pharm. Bull. 21,643-645. and Padmahaban, (Curcumin for malaria therapy, BBRC)

But the major problem for curcumin's use in therapy thus far has been it's poor bioavailability. In the view of the high lipophilic character of curcumin molecule, one would expect the body fat to contain a high proportion of bound curcumin. The poor absorption from intestine, coupled with the high degree of metabolism of curcumin in the liver and its rapid elimination in the bile, makes it unlikely that high concentrations of the substance would be found in the body long after ingestion. These pharmacokinetic properties of curcumin have been confirmed by using HPLC technique. Thus the systemic bioavailability of curcumin is low, 75% being excreted in the feces and only traces appeared in the urine (Wahlstrom et at., 1978 A study on the fate of curcumin in the rat. Acta Pharmacologica et Toxicologica 43, 86-92).

Due to the numerous therapeutic indications in which curcumin can be used, enhanced bioavailability of curcumin in the near future is likely to bring this promising natural product to the forefront of therapeutic agents for treatment of various human diseases. There have been attempts made in the prior art to increase the bioavailability of curcumin. To improve the bioavailability of curcumin, numerous approaches have been undertaken.

WO/2007/103435 provides curcuminoid compositions that exhibit enhanced bioavailability and is provided as microemulsion, solid lipid nanoparticles (SLN), microencapsulated oil or the like.

WO/2008/043157 provides compositions for modulating an immune response, which may be contained in one or more particles such as nanoparticles or microparticles. In some embodiments, the particle comprises a polymeric matrix or carrier, illustrative examples of which include biocompatible polymeric particles.

WO/2006/022012 describes a novel and stable solid dispersion of curcumin produced by dissolving curcumin together with polyvinylprrloidone in an alcoholic solvent and then spray-drying.

CN1736369 provides a curcumin oil emulsion and injection, wherein the emulsion comprises curcumin, oil, emulsifying agent and water.

Savita Bisht el al (Polymeric nanoparticle-encapsulated curcumin (“nanocurcumin”): a novel strategy for human cancer therapy, J Nanobiotechnology. 2007; 5: 3.) disclose polymeric nanoparticle encapsulated formulation of curcumin—nanocurcumin—utilizing the micellar aggregates of cross-linked and random copolymers of N-isopropylacrylamide (NIPAAM), with N-vinyl-2-pyrrolidone (VP) and poly(ethyleneglycol)monoacrylate (PEG-A).

Curcumin delivered through liposomes has been shown to be effective in suppressing pancreatic carcinoma growth in murine xenograft models. (Li L, Braiteh FS, Kurzrock R. Cancer 2005;104:1322-31). But the drawback of any liposomal prepration is its instability under physiological conditions and under storage conditions (T. Ruysschaert, M. Germain, J. F. Gomes, D. Fournier, G. B. Sukhorukov, W. Meier and M. Winterhalter, IEEE Trans. Nanobiosci. 2004, 3, 49-55 & Sukhorukov, A. Fery and H. Mohwald, Intelligent micro- and nanocapsules, Prog. Polym. Sci. 2005, 885-897). Repeated administration of liposome may have some effect on age related diseases including cardiovascular diseases, malignancy and autoimmune diseases. (G. Fernandes, Current Opinion in Immunology, 1989-90,2, 275-281).

N-isopropylacrylamide, N-vinyl-2-pyrrolidone and poly(ethyleneglycol)monoacrylate have also been tried for the preparation of curcumin nanoparticles in prio art. A study conducted by J Sakamoto and K Hashimoto using rats shows that oral administration of N-isopropylacrylamide to rats , in drinking water for 45 days can induce severe signs of neuropathy as well as body weight loss (J Sakamoto et al, Archives of toxicology, 1985, 57, 282-4.) Another study conducted by K Hashimoto, J Sakamoto and H Tanii using acrylamide and related compounds showed that N-isopropylacrylamide when given orally to mice caused neurotoxicity and testicular atrophy. (Archives of toxicology, 1981, 47, 179-89). Therefore, long term use of such nano particles can not be recommended without toxicity studies.

The curcumin nanoparticles and chitosan nanoparticles coated with curcumin when fed orally to mice showed improved bioavailability of curcumin and cured Plasmodium yoelii infected mice.

### SUMMARY OF THE INVENTION

The present invention provides curcumin nanoparticles made out of curcumin only and curcumin bound to chitosan nanoparticles. The bioavailability of curcumin from such nanoparticles, in particular, was tested by determining it's ability to cure Plasmodium yoelii infection in mice. Bioavailability of curcumin in mice from the invented formulations increased by 10 fold. Curcumin from said nanoparticles was also seen to persist in mice for a longer duration as compared to curcumin administered in olive oil thereby increasing the efficacy of the treatment.

### DESCRIPTION OF THE ACCOMPANYING DRAWINGS

FIG. 1.1 DLS of curcumin bound to Chitosan nano particles

FIG. 1.2 DLS of Curcumin nano particles

FIG. 1.3 Zeta potential of different nano particles

FIG. 1.4 Viscocity of different nano particles

FIG. 2.1 TEM picture of Chitosan nano particles

FIG. 2.2 TEM Picture of curcumin bound to chitosan nano particles

FIG. 2.3 TEM Picture of curcumin nano particles

FIG. 3 Increase in bioavailability of curcumin when delivered bound to chitosan nano particle, or as nano particle or delivered through olive oil

FIG. 4.1 Parasitemia in Infected Control Group

FIG. 4.2 Parasitemia in Olive oil Control Group

FIG. 4.3 Parasitemia Chitosan nano particle Control Group

FIG. 4.4 Parasitemia in Curcumin in olive oil Group

FIG. 4.5 Parasitemia in Curcumin bound to chitosan nanoparticle Group

FIG. 4.6 Parasitemia in Curcumin nanoparticle Group

FIG. 5.1 FACS analysis of RBC taken from uninfected mouse not fed with curcumin nanoparticles

FIG. 5.2 FACS analysis of RBC taken from Normal mouse fed with curcumin nanoparticles

FIG. 5.3 FACS analysis of RBC taken from infected mouse fed with curcumin nanoparticles

FIG. 5.4 FACS analysis data showing curcumin fluorescence intensity of uninfected and infected RBC

FIG. 5.5 Accummulation of curcumin in infected RBC taken from mouse with different parasitemia who were fed with curcumin nanoparticles

FIG. 5.6 Confocal microscopy showing the accumulation of curcumin in erythrocytes of uninfected mice fed with curcumin nanoparticles

FIG. 5.7 Confocal microscopy showing the accumulation of curcumin in erythrocytes of nfected mice fed with curcumin nanoparticles

FIG. 6 In vivo inhibition of hemozoin synthesis in P. yoelii infected mice by feeding chloroquinine in normal saline or curcumin bound to chitosan nanoparticles (hemozoin concentration is measured in terms of dissociated home)

FIG. 7 TUNEL assay showing apoptosis in isolated parasite from infected mice fed with curcumin bound to chitosan nanoparticles.
A. Control mice receiving no treatment shows very little apoptosis (0.18%).
B. Infected mice given only chitosan nanoparticles orally showed 4.6% apoptosis.
C. Infected mice given only curcumin through olive oil orally showed 4.47% apoptosis.
D. Infected mice given curcumin bound to chitosan nanoparticles orally showed 9.64% apoptosis.

FIG. 8 Summary of the TUNEL assay described in FIG. 7

FIG. 9.1 FTIR spectra of chitosan

FIG. 9.2 FTIR spectra of Chitosan nanoparticles

FIG. 9.3 FTIR spectra of Curcumin

FIG. 9.4 FTIR spectra of Curcumin nanoparticles

FIG. 9.5 FTIR spectra of Curcumin bound to chitosan nanoparticles

FIG. 10.1 Matrix Assisted Laser Desorption Ionization (MALDI) profile of Curcumin indicating the presence of the three curcuminoids in the sample i.e curcumin (mass 369) , Demethoxycurcumin (mass 339) and Bisdemethoxycurcumin (mass 309)

FIG. 10.2 MALDI profile of Curcumin nanoparticles indicating the presence of the same molecules ie curcumin (mass 369), Demethoxy curcumin (339) and Bisdemethoxy curcumin (309).

FIG. 10.3 HPLC profile of Curcumin separated on a C-18 column using an isocratic solvent system: acetonitrile: methanol: water: acetic acid::41: 23: 36:1.

FIG. 10.4 HPLC profile of Curcumin nanoparticles separated on a C18 column after dissolving in ethanol using the same isocratic solvent system for separation. It shows the same profile as curcumin.

FIG. 11 Effect of oral intake of curcumin and nanocurcumin on fasting glucose level of human volunteers.

FIG. 12.1 Effect of oral intake of curcumin and nanocurcumin on Urea level of human Volunteers

FIG. 12.2 Effect of oral intake of curcumin and nanocurcumin on creatinine level of human volunteers

FIG. 12.3 Effect of oral intake of curcumin and nanocurcumin on potassium level of human volunteers (Only Seven Volunteers)

FIG. 13.1 Effect of oral intake of curcumin and nanocurcumin on Total cholesterol level of human volunteers

FIG. 13.2 Effect of oral intake of curcumin and nanocurcumin on HDL cholesterol level of human volunteers

FIG. 13.3 Effect of oral intake of curcumin and nanocurcumin on LDL cholesterol level of human volunteers

FIG. 13.4 Effect of oral intake of curcumin and nanocurcumin on Triglycerides level of human volunteers

FIG. 13.5 Effect of oral intake of curcumin and nanocurcumin on sodium level of human Volunteers.(Only Seven Volunteers)

FIG. 14.1 Effect of oral intake of curcumin and nanocurcumin on Hemoglobin level of human volunteers

FIG. 14.2 Effect of oral intake of curcumin and nanocurcumin on RBC count level of human volunteers

FIG. 15.1 Effect of oral intake of curcumin and nanocurcumin on SGPT level of human volunteers

FIG. 15.2 Effect of oral intake of curcumin and nanocurcumin on SGOT level of human volunteers

FIG. 15.3 Effect of oral intake of curcumin and nanocurcumin on ALP level of human volunteers

FIG. 15.4 Effect of oral intake of curcumin and nanocurcumin on total Bilirubin level of human volunteers

FIG. 15.5 Effect of oral intake of curcumin and nanocurcumin on albumin level of human volunteers

FIG. 16.1 Effect of oral intake of curcumin and nanocurcumin on globulin level of human volunteers

FIG. 16.2 Effect of oral intake of curcumin and nanocurcumin on eosinophiles level of human volunteers

FIG. 16.3 Effect of oral intake of curcumin and nanocurcumin on neutrophils level of human volunteers

FIG. 16.4 Effect of oral intake of curcumin and nanocurcumin on platelet count level of human volunteers

### DETAILED DESCRIPTION

The term “organic acid” refers to any organic compound with acidic properties. Representative examples include but are not limited to acetic acid, citric acid and propionic acid.

The term “alcohol” refers to any organic compound in which a hydroxyl group (—OH) is bound to a carbon atom of an alkyl or substituted alkyl group. Representative examples include but are not limited to ethanol, methanol and propanol.

In the present invention curcumin nanoparticles were prepared. In one embodiment, nanoparticles were also made out of the mucoadhesive biopolymer chitosan to deliver curcumin orally into mice. Curcumin was loaded on the surface of the chitosan nanoparticles. This more efficient delivery vehicle ensured enhanced bioavailability and sustained circulation of curcumin in the blood compared to oral delivery of curcumin alone dissolved in olive oil. Importantly, this procedure does not involve any chemical modification of curcumin and binding occurs due to the availability of hydrophobic pockets on the surface of the chitosan nanoparticles. Chitosan nanoparticles not only improved the bioavailability of curcumin but also increased its stability.

The process involved dissolving a clear solution of Chitosan in an organic acid by heating the mixture at 50° C.-80° C. The mixture was rapidly cooled to 4° C.-10° C. and this process was repeated till a clear solution was obtained. The solution was then heated at 50° C.-80° C. and sprayed under pressure into water kept stirring at 2° C.-10° C. This solution containing the Chitosan nanoparticles was stored for further use. The chitosan nanoparticles can be concentrated by centrifugation at slow speed. A clear solution of curcumin was prepared in alcohol. This curcumin solution was added under pressure to vigorously stirred aqueous suspension of chitosan nanoparticles in an organic acid and the resulting suspension was stirred overnight at room temperature to load curcumin on the chitosan nanoparticle. For the release study, curcumin-chitosan nanoparticles suspension was centrifuged and the pellet was resuspended with equal volume of water and was centrifuged two more times with purified water to remove unbound curcumin from the nano particles.

Accordingly in one embodiment the process involved dissolving a clear solution of 0.025%-1% (w/v) Chitosan in 0.1% -10% or more, preferably 0.5%-1% aqueous acetic acid by heating the mixture at 50° C.-80° C. The mixture was rapidly cooled to 4° C.-10° C. and this process was repeated till a clear solution was obtained. The solution was then heated at 50° C.-80° C. and sprayed under pressure into water kept stirring at 200-1400 rpm at 4° C.-10° C. This solution containing the Chitosan nanoparticles was stored for further use. The chitosan nanoparticles can be concentrated by centrifugation at slow speed. A clear solution of 0.1-1.0 g of curcumin was prepared in 100-1000 ml of ethanol. This curcumin solution was added under pressure to vigorously stirred aqueous suspension of chitosan nanoparticles in 0.1%-10% or more, preferably 0.25% -1% acetic acid and the resulting suspension was stirred overnight at room temperature to load curcumin on the chitosan nanoparticle. For the release study, curcumin-chitosan nanoparticles suspension was centrifuged and the pellet was resuspended with equal volume of water and was centrifuged two more times with purified water to remove unbound curcumin from the nano particles.

In the case of curcumin bound to chitosan nanoparticles, the concentrations of both chitosan and curcumin affect the size of the nanoparticle.

In another embodiment of the invention, curcumin nanoparticles were prepared by dissolving curcumin in alcohol and then spraying the solution kept at 25° C.-40° C. under nitrogen atmosphere and high pressure into an organic acid solution kept stirring at room temperature. Stabilizers or surfactants were not used and the finished product entirely consisted of curcumin in the form of nanoparticles.

Accordingly, curcumin nanoparticles were prepared by dissolving 0.1-1 g curcumin in 100-1000 ml 5%-100% of ethanol, preferably absolute ethanol and then spraying the solution kept at 25° C.-40° C. under nitrogen atmosphere and high pressure into 0.1%-10% or more, preferably 0.25%-0.1% aqueous acetic acid solution kept stirring at room temperature. Stabilizers or surfactants were not used and the finished product entirely consisted of curcumin in the form of nanoparticles.

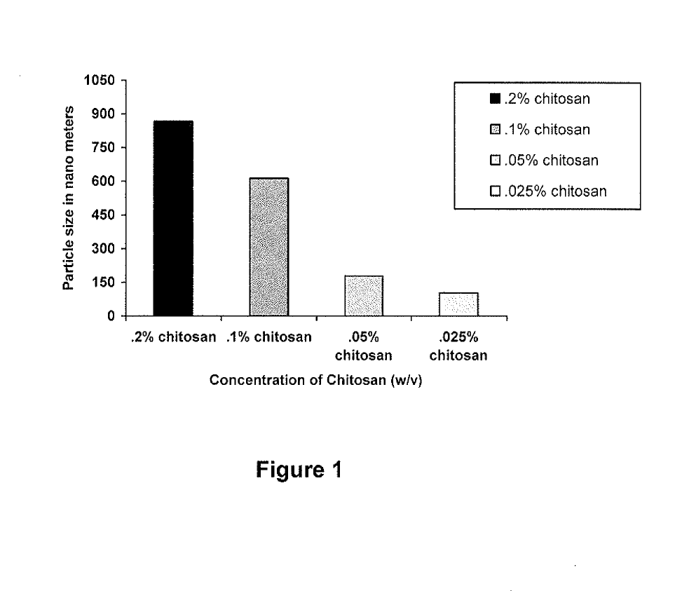

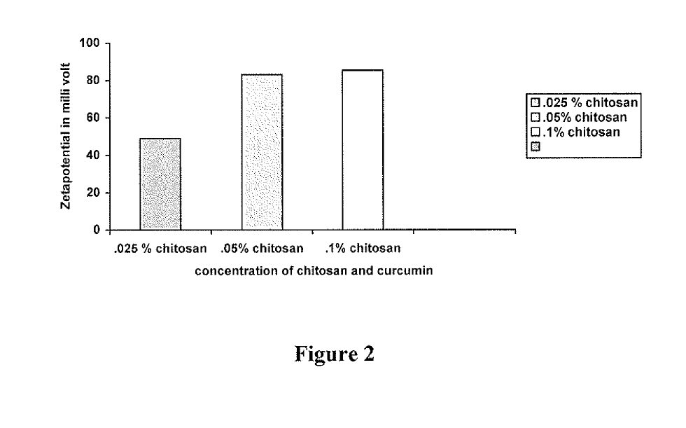

Dynamic light scattering (DLS) (Malvern, Autosizer 4700) was used to measure the hydrodynamic diameter and size distribution (polydispersity index, PDI=_{—}μ2_{—}/Γ2). Chitosan loaded curcumin nanoparticles of size 43 nm to 325 nm, preferably 43 nm to 83nm, and curcumin nanoparticles of size 50 nm to 250 nm, preferably 50 nm to 135 nm were obtained as indicated in FIGS. 1.1 & 1.2. The zeta potential and viscosity of nanoparticles was measured on a zeta potential analyzer (Brookhaven, USA) and a Viscometer FIGS. 1.3 & 1.4. Particle morphology was examined by transmission electron microscopy (TEM) (Hitachi, H-600). FIGS. 2.1-2.3

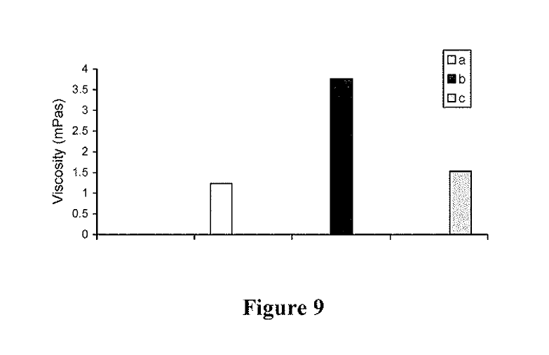

Nanoparticles were dried in a vacuum dessicator and their FTIR were taken with KBr pellets using the Nicolet Magna 550 IR Spectrometer FUR spectra of Chitosan nano particle has similar absorbance pattern as that of chitosan. (FIGS. 9.1-9.2). Similarly the FTIR spectra of curcumin and curcumin nano particles were similar indicating that curcumin was not chemically modified when it is converted into nanoparticles (FIGS. 9.3-9.4). The FTIR spectra of curcumin bound to chitosan nano particles as expected had all the features of chitosan and curcumin indicating the curcumin is not altered in the process of binding to chitosan nano particles (FIG. 9.5).

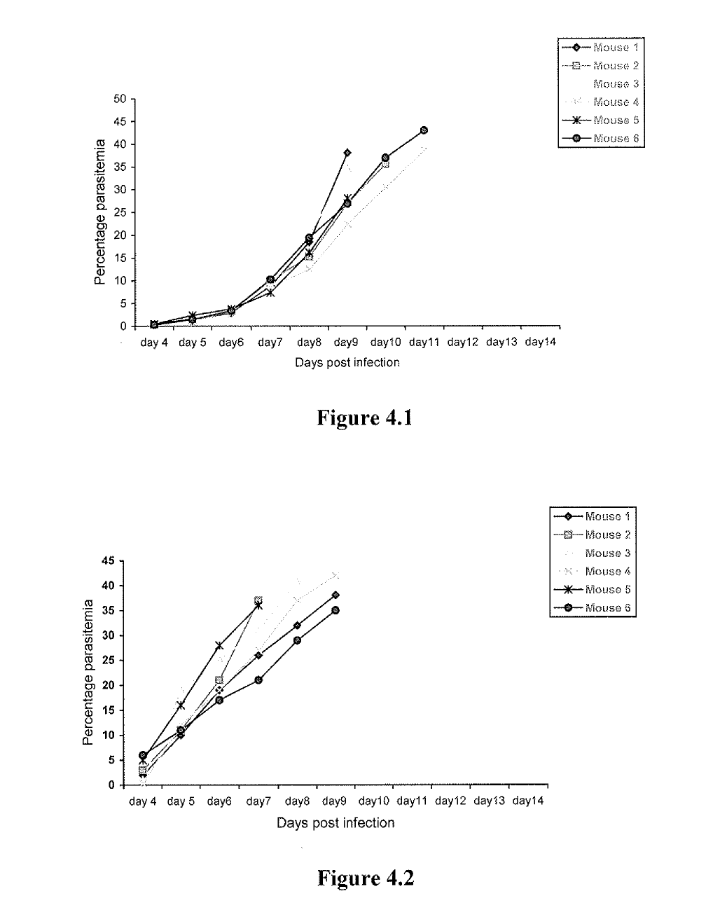

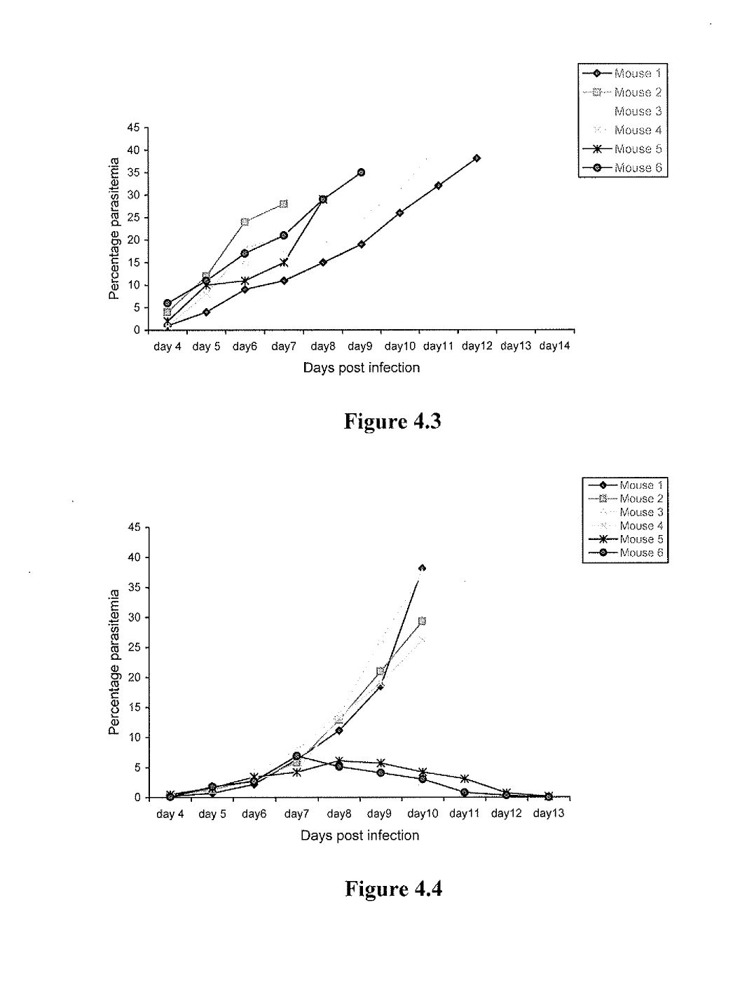

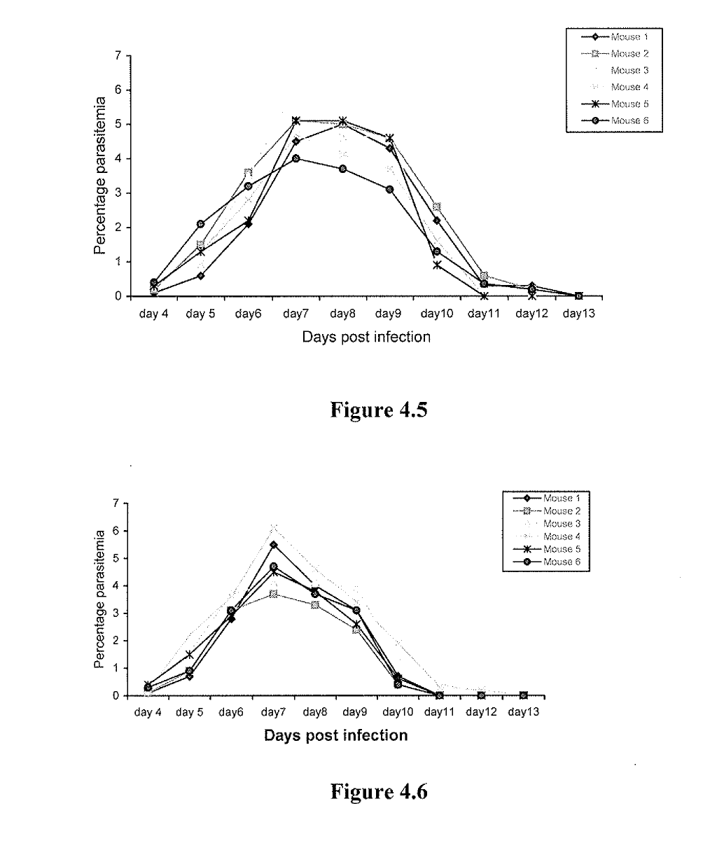

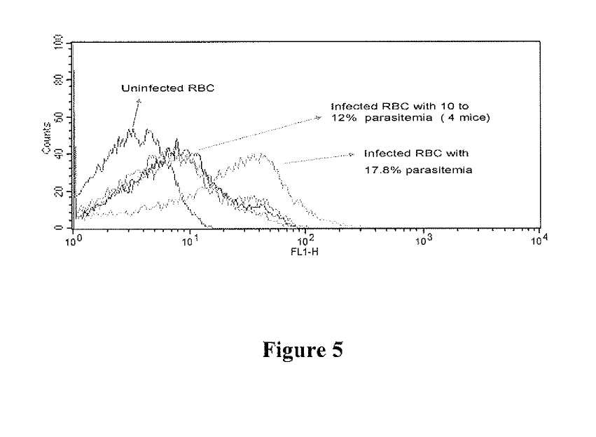

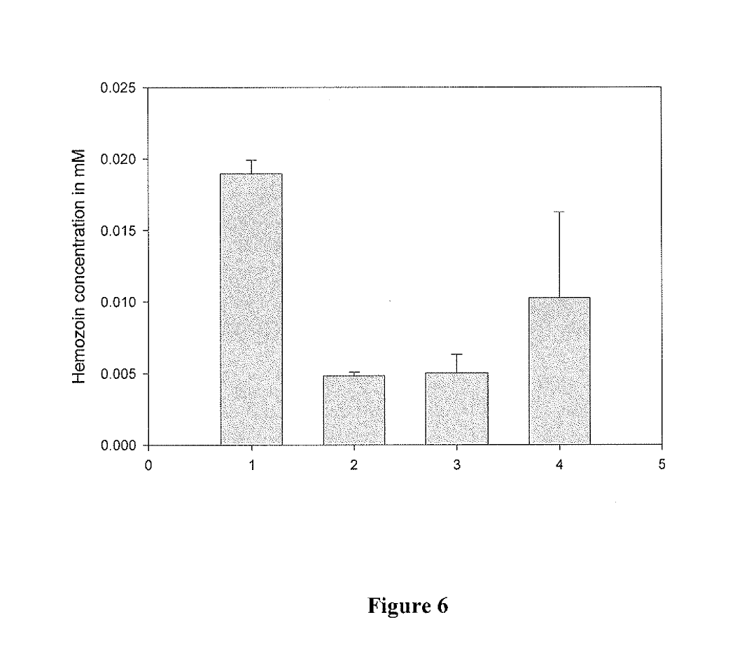

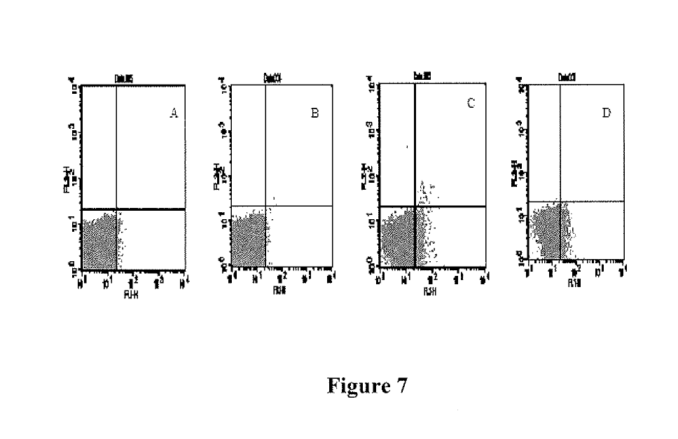

Both the curcumin nanoparticle and the curcumin bound to chitosan nanoparticle cured 100% of the mice infected with a lethal strain of Plasmodium yoelii parasite compared to infected untreated control where all animals died FIG. 4.1-4.6. The cured mice populations survived for at least 100 days and were resistant to subsequent reinfection in 100% cases. It was found that curcumin preferentially accumulated inside the infected erythrocytes, the quantity increasing with increase of parasite load in the erythrocyte FIG. 5.5. Confocal microscopy revealed that curcumin was bound to the parasite FIG. 5.7. Just like chloroquine, curcumin inhibited hemozoin formation in vivo which the parasite makes to avoid the toxicity of heme (FIG. 6.)

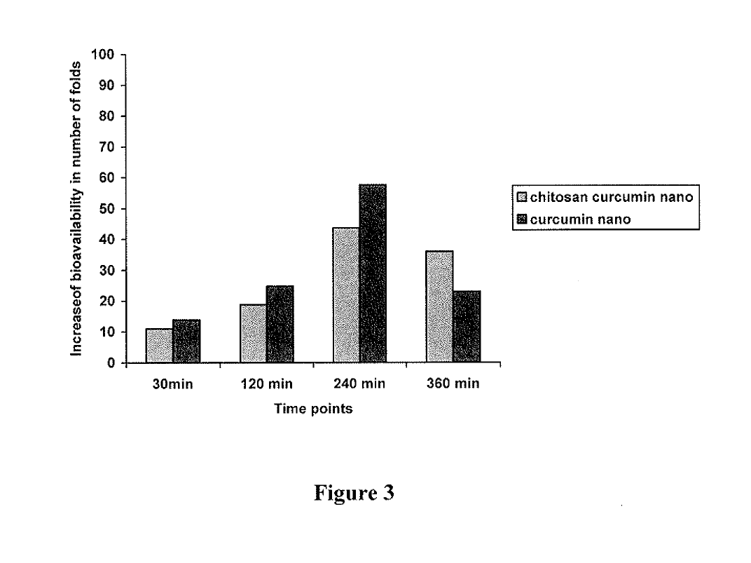

Curcumin nanoparticles and curcumin bound to chitosan nanoparticles demonstrated a 10 fold increase in bioavailability of curcumin (FIG. 3.) and they were efficient in killing malaria parasite in vivo in mice. FIG. 4.5-4.6.

The scope of the invention extends to all possible pharmacological uses of curcumin such as use of curcumin in the treatment of cancers, diseases involving an inflammatory reaction, alzheimer's disease, cholesterol gall stones, diabetes, alcohol and drug induced liver diseases, parasitic infestation, malaria and other parasitic diseases, neurological disorders and all other diseases that can be treated or managed using curcumin.

### EXAMPLE 1

### Preparation of Curcumin Bound to Chitosan Nanoparticles

1.1 Preparation of Chitosan Nanoparticles

A clear solution of 0.2% Chitosan (w/v) in 1% acetic acid was prepared by heating the mixture to 75° C. The mixture was rapidly cooled to 4° C. and this process was repeated several times till a solution of chitosan was obtained. This solution was then heated to 75° C. again and sprayed under pressure into water kept stirring very rapidly at 4° C. This ensured production of uniformly dispersed chitosan nanoparticles which can be concentrated by centrifugation

1.2 Loading Curcumin on Chitosan Nanoparticles

A clear solution of 1 gm of curcumin in 1000 ml of absolute ethanol was added under pressure to vigorously stirred aqueous suspension of chitosan nanoparticles in 1% acetic acid and the resulting suspension was stirred overnight at 200 -1400 rpm at room temperature to load curcumin on the chitosan nanoparticle.

### EXAMPLE 2

### Preparation of Curcumin Nanoparticles

1 gm of curcumin was dissolved in 1000 ml of absolute ethanol. The solution was kept at 40° C. and then sprayed under nitrogen atmosphere and high pressure into 0.1% aqueous acetic acid solution which was kept stirring at 200 -1400 rpm at room temperature. This lead to the production of uniformly dispersed curcumin nanoparticles. The particle size can be controlled by varying the pressure at which curcumin solution is sprayed into 0.1% aqueous acetic acid kept at different temperatures (25° C. -40° C.).

### EXAMPLE 3

### Biophysical Characterization of Nanoparticles

3.1 Particles Size Measurement by Dynamic Light Scattering

Dynamic light scattering (DLS) was used to measure the hydrodynamic diameter and size distribution (FIG. 1.1-1.2). Dynamic light scattering (DLS) experiments were performed (scattering angle=90°, laser wavelength=632.8 nm) on a 256 channel Photocor-FC (Photocor Inc., USA) that was operated in the multi-tau mode (logarithmically spaced channels). During the titration process, a few milliliters of the sample was drawn from the reaction beaker and loaded into borosilicate cylindrical cell (volume=5 ml) and DLS experiment performed. The data was analyzed both in the CONTIN regularization and discrete distribution modes (multi-exponential). The CONTIN software generates the average relaxation time of the intensity correlation function, which is solely related to Brownian dynamics of the diffusing particles for dilute solutions. The intensity correlation data was force fitted to a double-exponential function without success. Thus, we have relied on a single exponential fitting (with polydispersity) and the chi-squared values were>90% consistently for all the correlation data. This yielded the apparent translational diffusion coefficient values. Correspondingly, the apparent hydrodynamic radii, Rₕ of the particles, at room temperature (°C.) were determined from the knowledge of translational diffusion coefficient D_{Γ}. These values were used in Stoke-Einstein equation, D=k_{B}Γ/f with the translational friction coefficient, f=6πη₀Rₕ, where k_{B} is Boltzmann constant, and n₀ is solvent viscosity.

3.2 Electrophoresis Studies

Electrophoretic mobility measurements were performed on the prepared nanoparticles (FIG. 1.3). The instrument used was Zeecom-2000 (Microtec Corporation, Japan) zeta-sizer that permitted direct measurement of electrophoretic mobility and its distribution. In all our measurements the migration voltage was fixed at 25 V. The instrument was calibrated against 10⁻⁴ M AgI colloidal dispersions. All measurements were performed in triplicate.

3.3 Particle Morphology by Transmission Electron Microscopy

Particle morphology was examined by transmission electron microscopy (TEM) (Hitachi, H-600). Samples were immobilized on copper grids. They were dried at room temperature, and subsequently examined using transmission electron microscope after staining with uranyl acetate (FIG. 2.1-2.3).

### EXAMPLE 4

### Evidence of Binding of Chitosan Nanoparticles with Curcumin

Chitosan nanoparticles and Chitosan nanoparticles loaded with curcumin were separated from suspension and were dried., and their FTIR was recorded with KBr pellets on Nicolet, Magna-550 spectrum. HPLC was performed after extracting curcumin from the nanosuspension. The particles were collected after high centrifugation and washed several times till the presence of curcumin was not detected in the supernatant by spectroscopic measurnent (absorbance recorded at 429 nm against ethanol). Curcumin was extracted from the pellet by the extraction solvent consisting of ethyl acetate and isopropanol (9:1). The upper organic layer was dried under nitrogen atmosphere. It was then reconstituted in ethanol and absorbance was recorded at 429 nm against ethanol as blank.

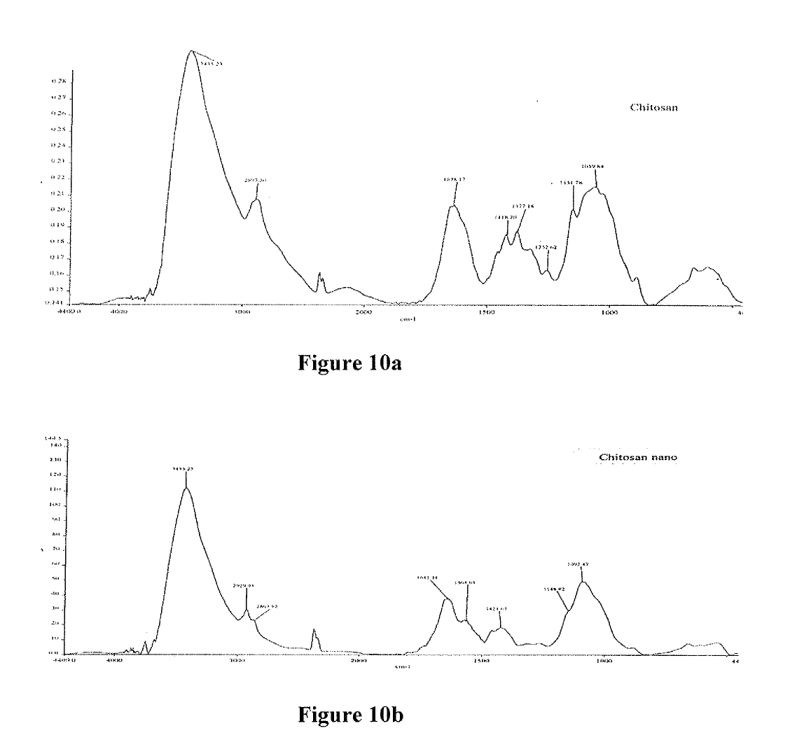

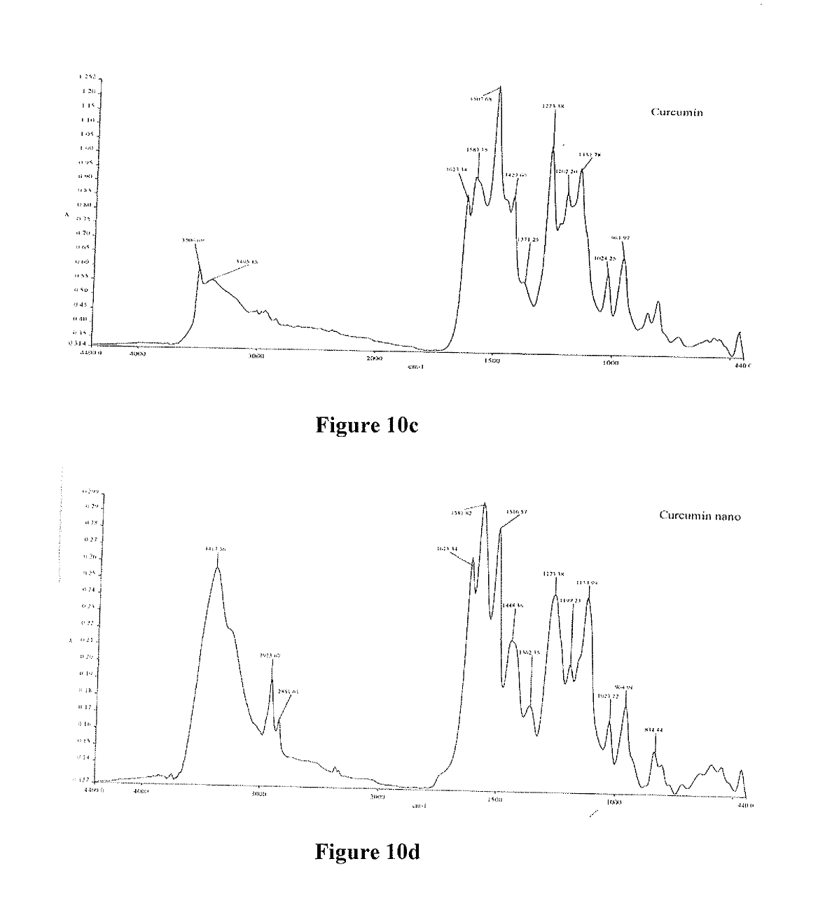

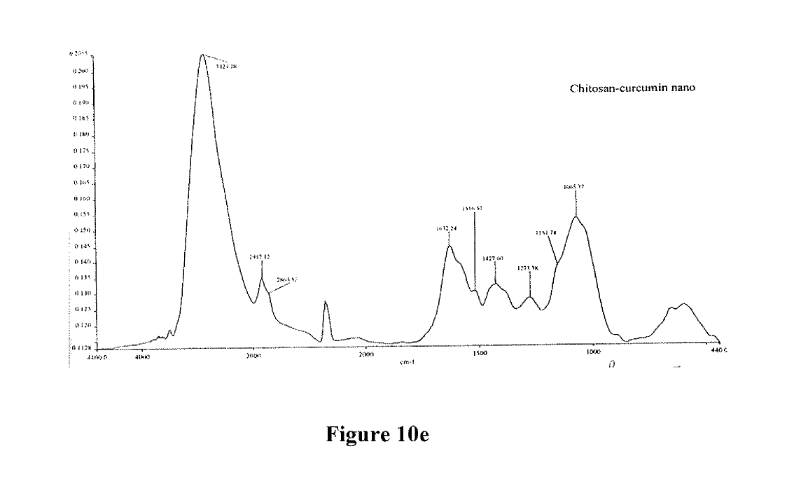

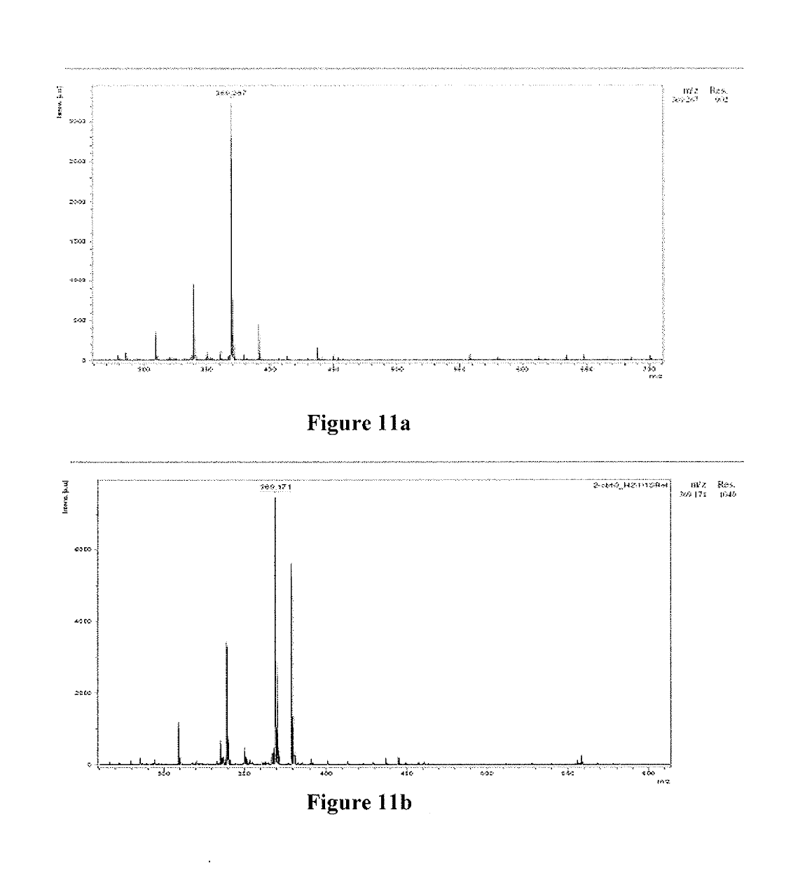

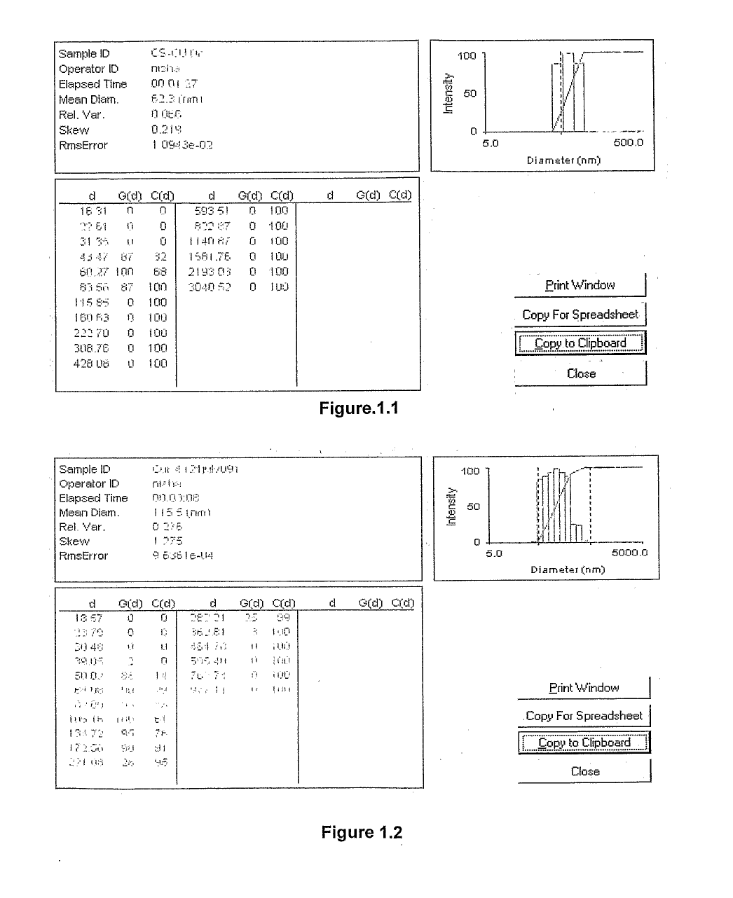

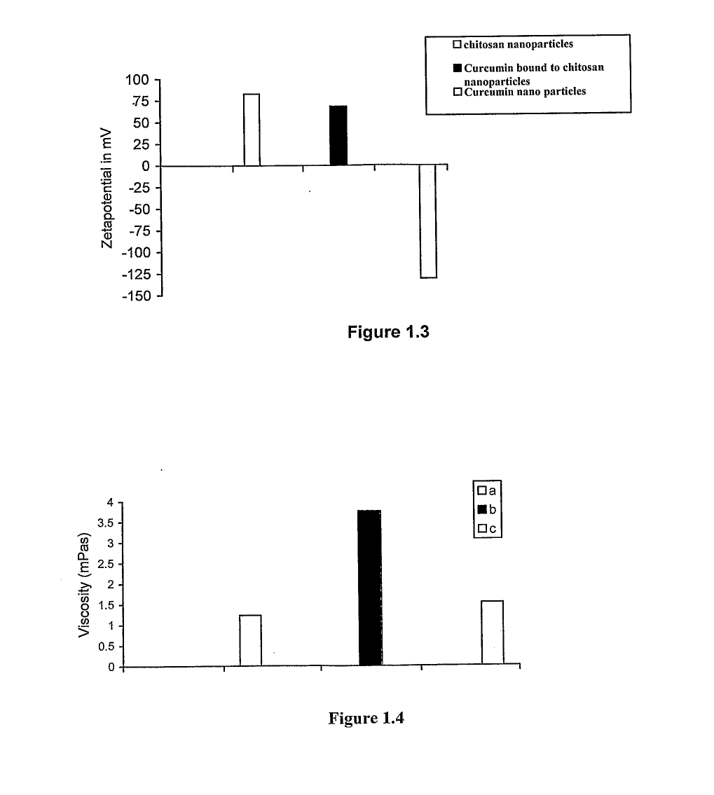

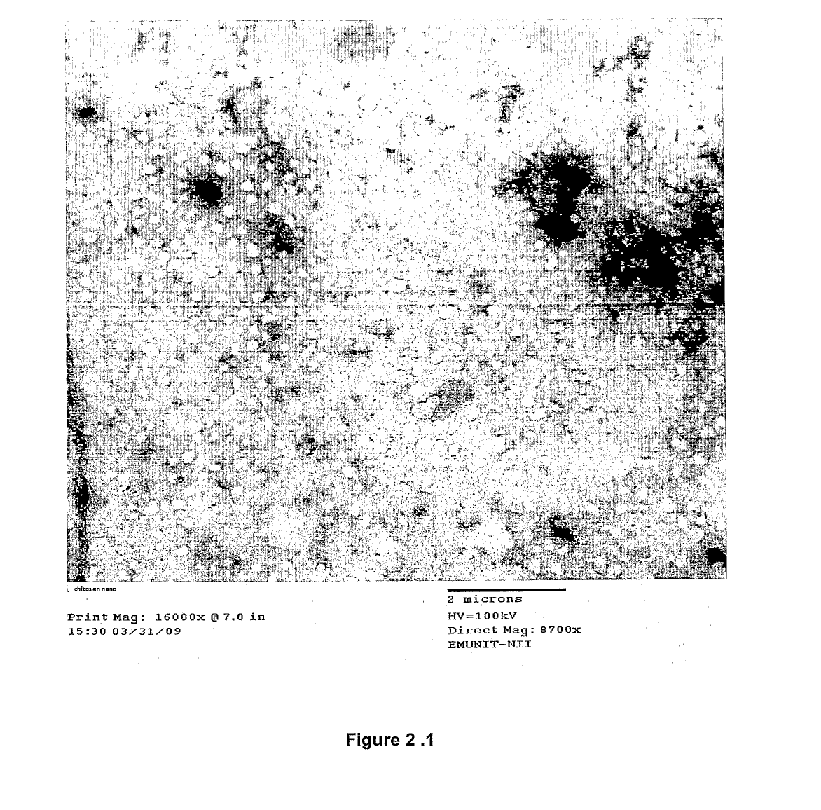

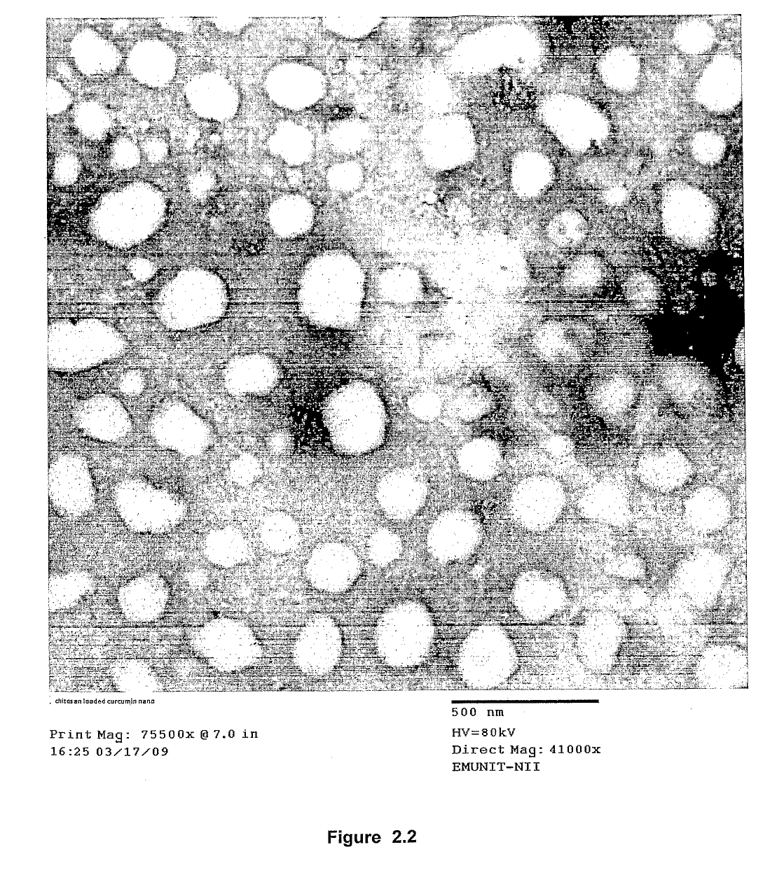

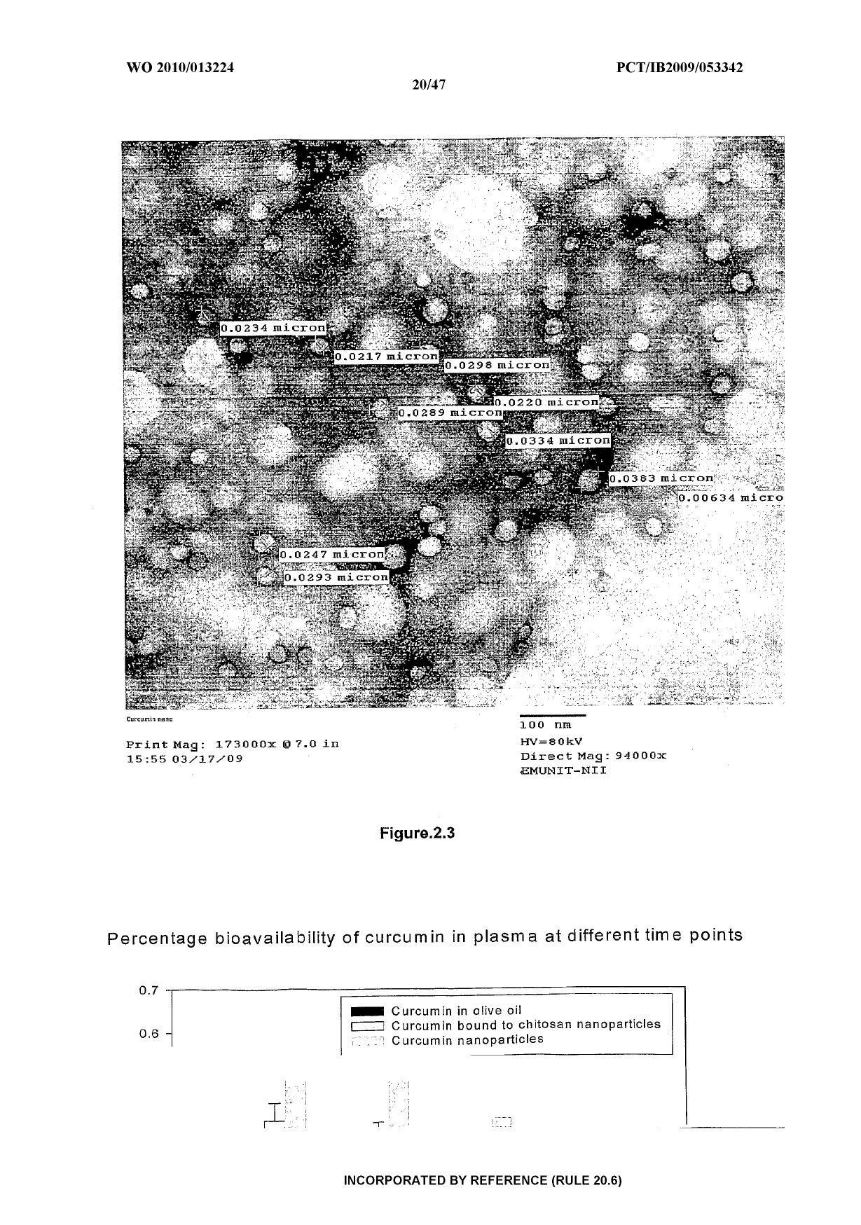

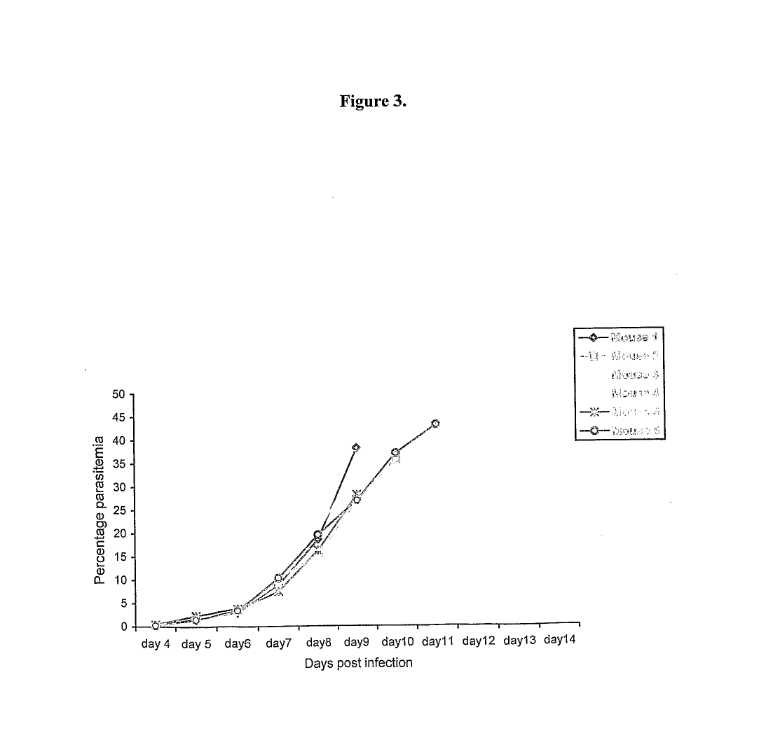

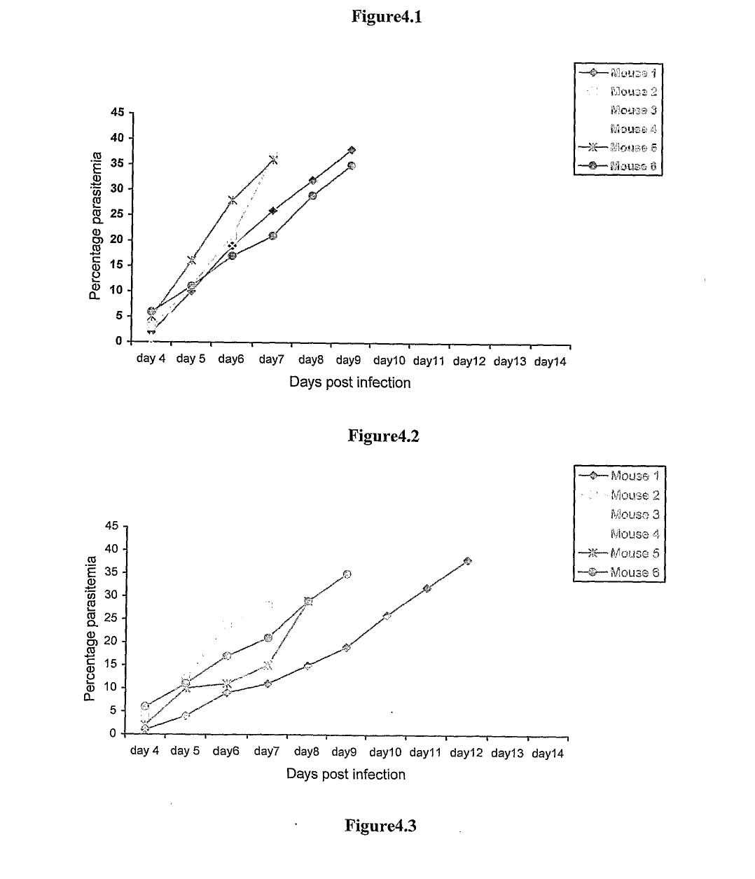

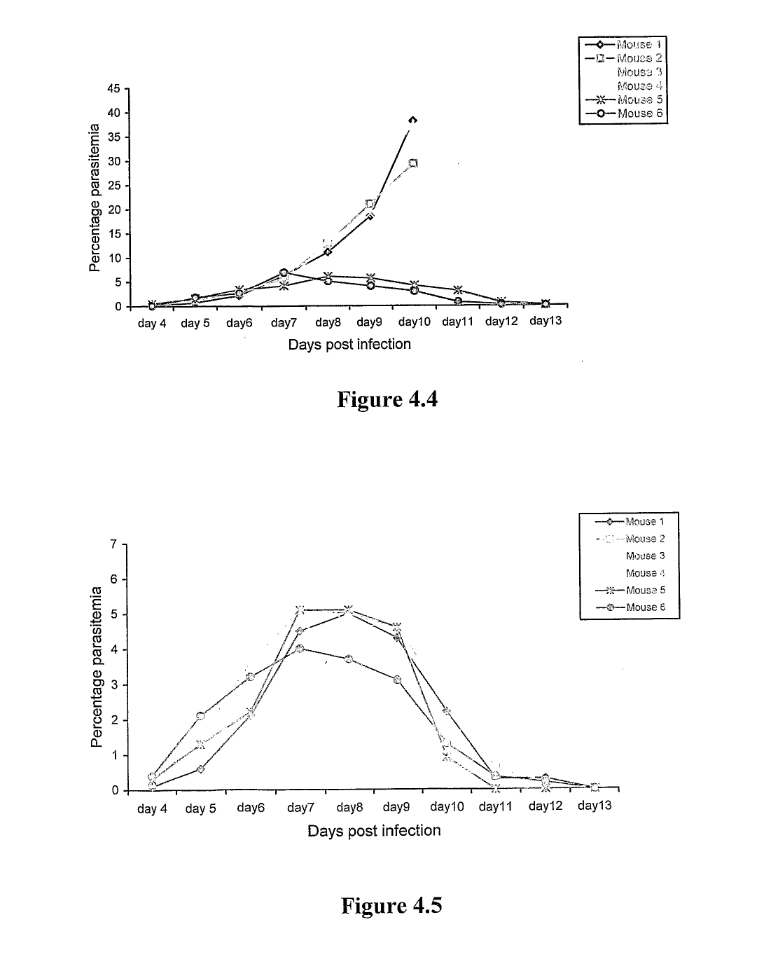

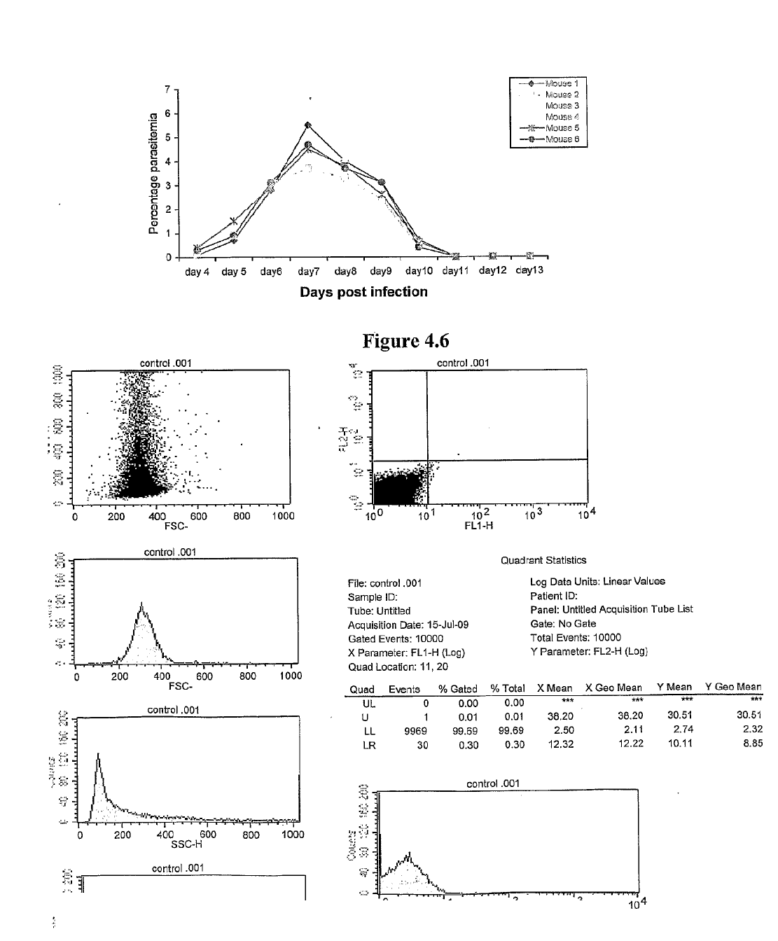

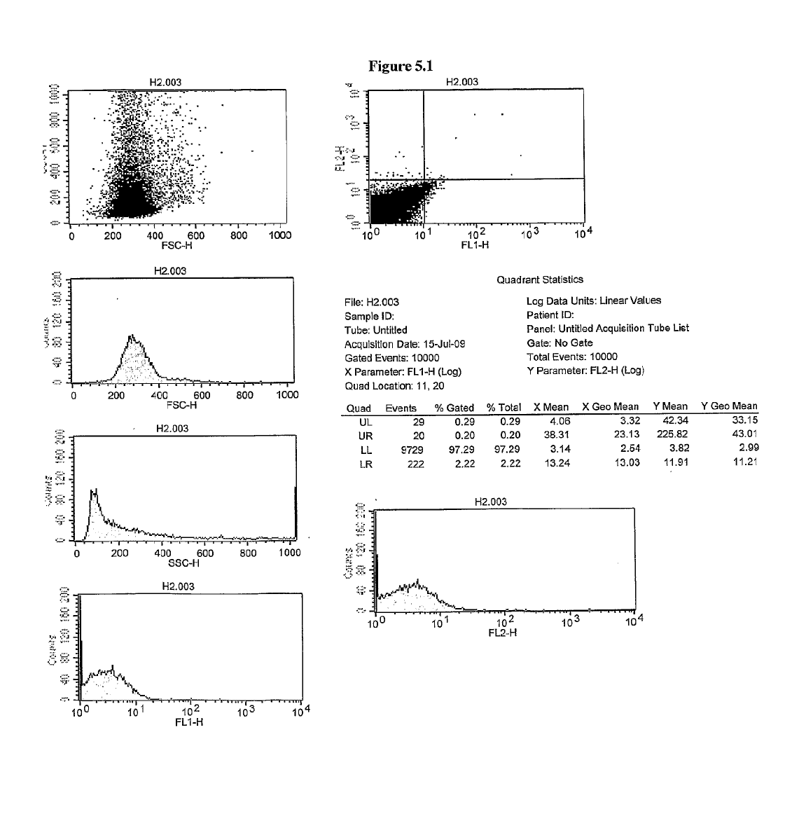

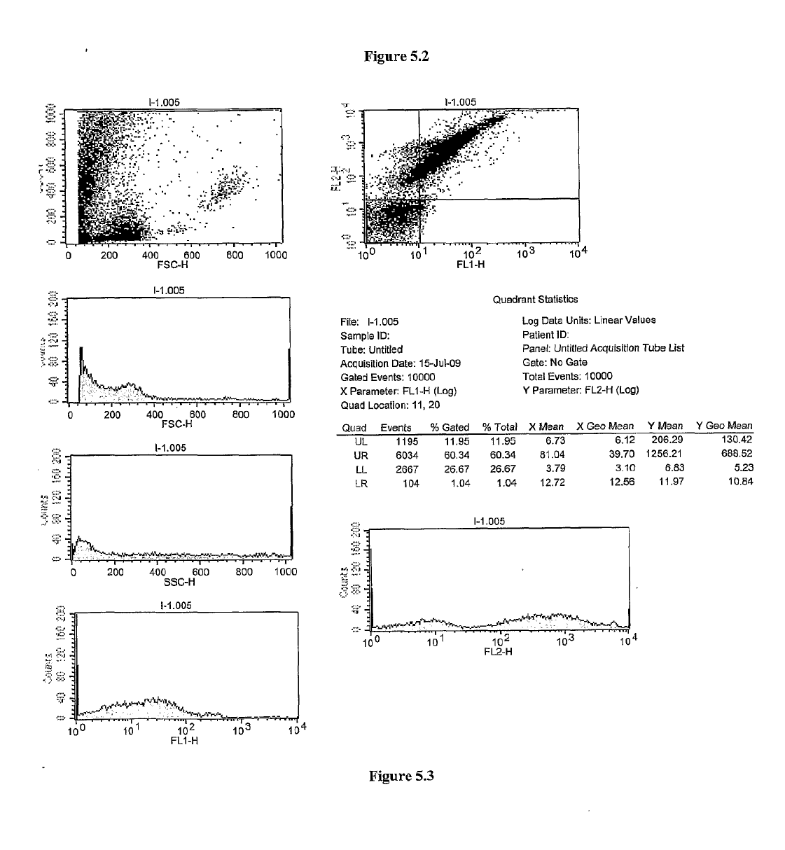

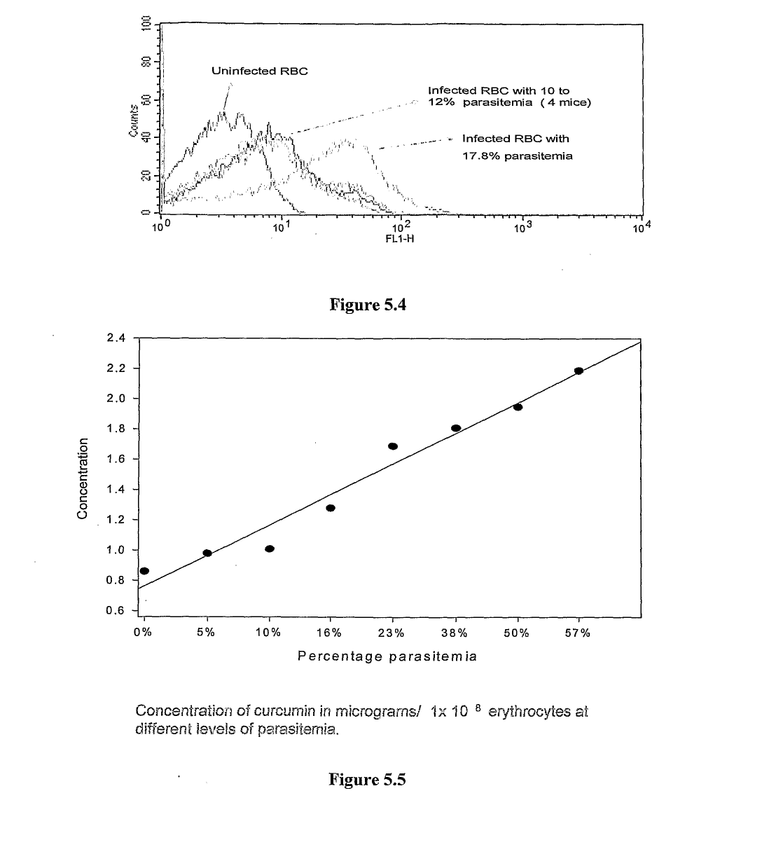

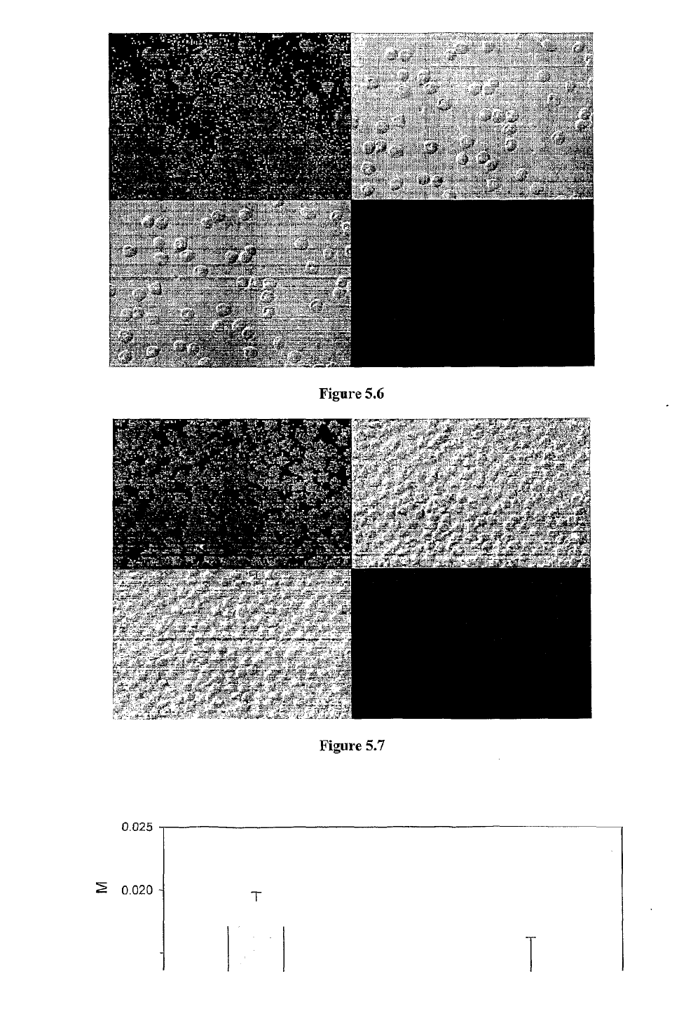

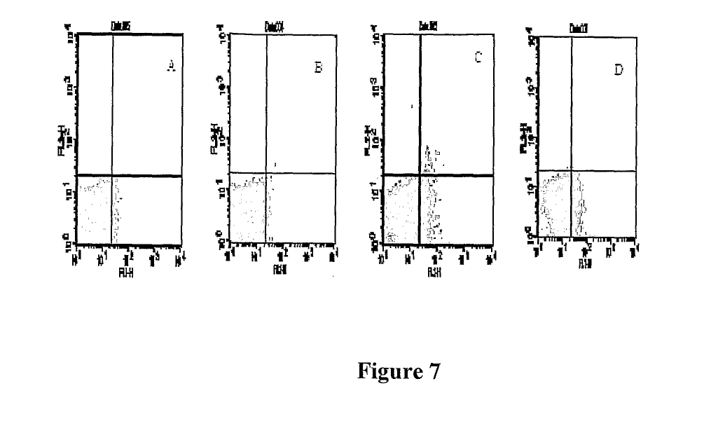

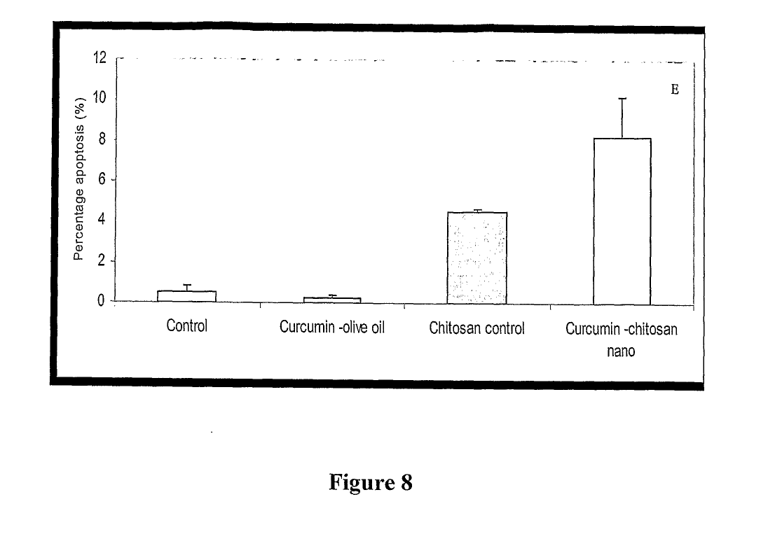

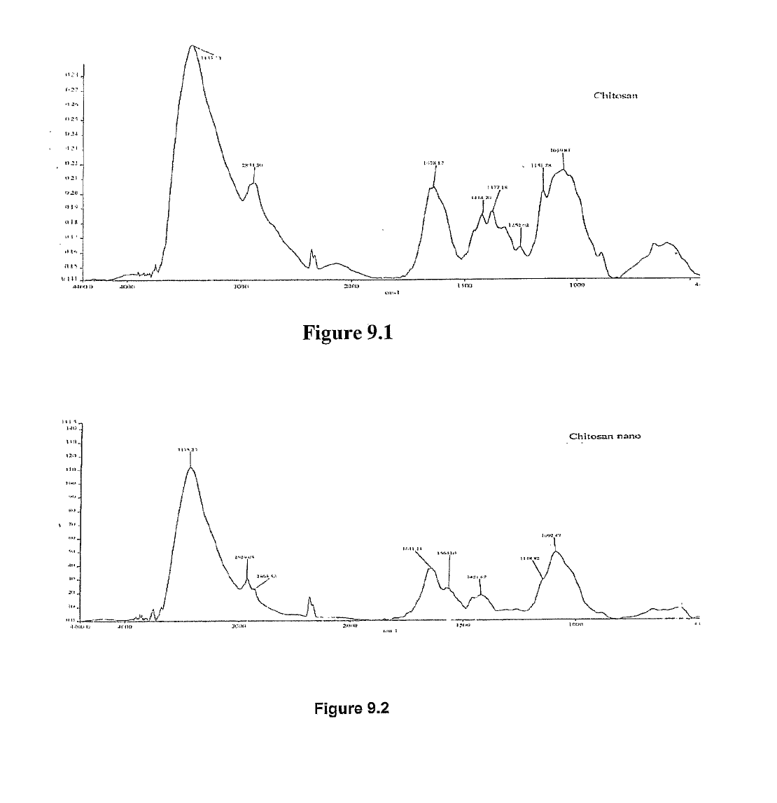

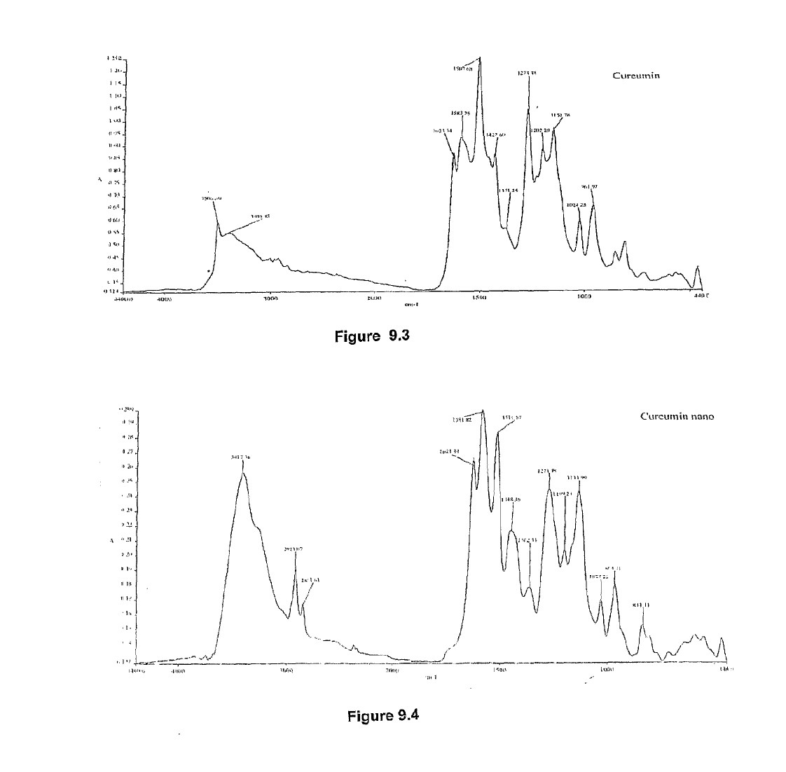

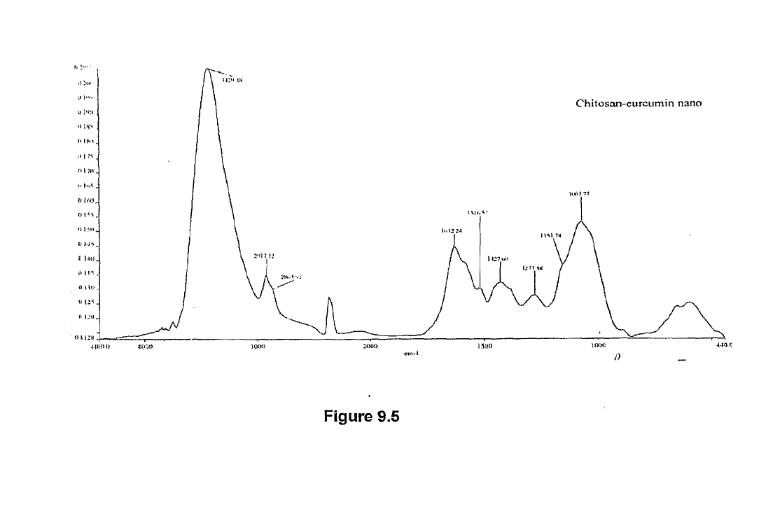

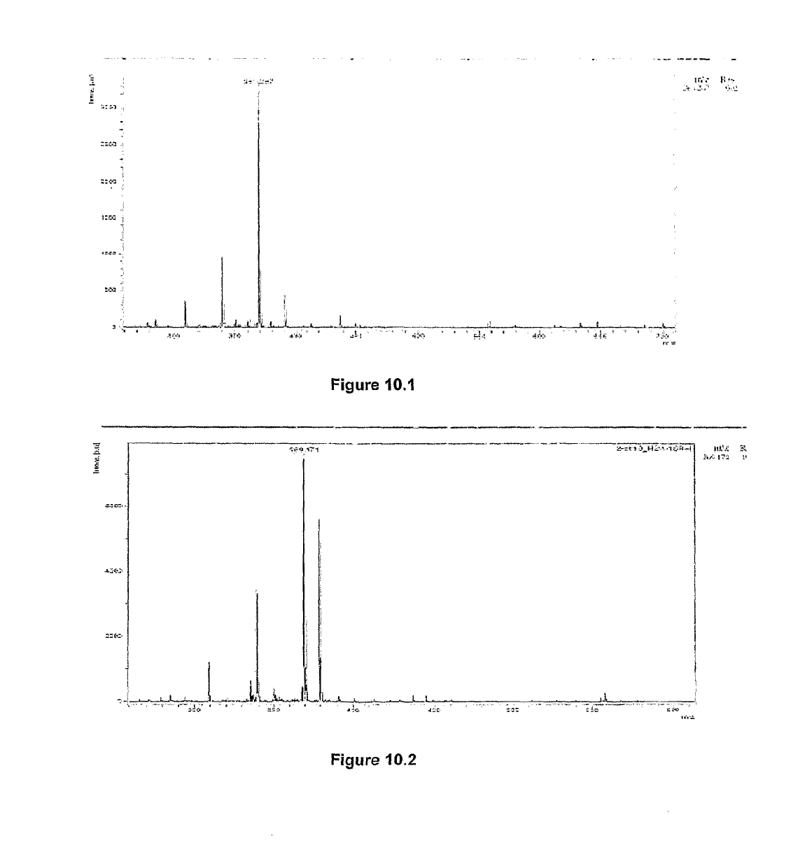

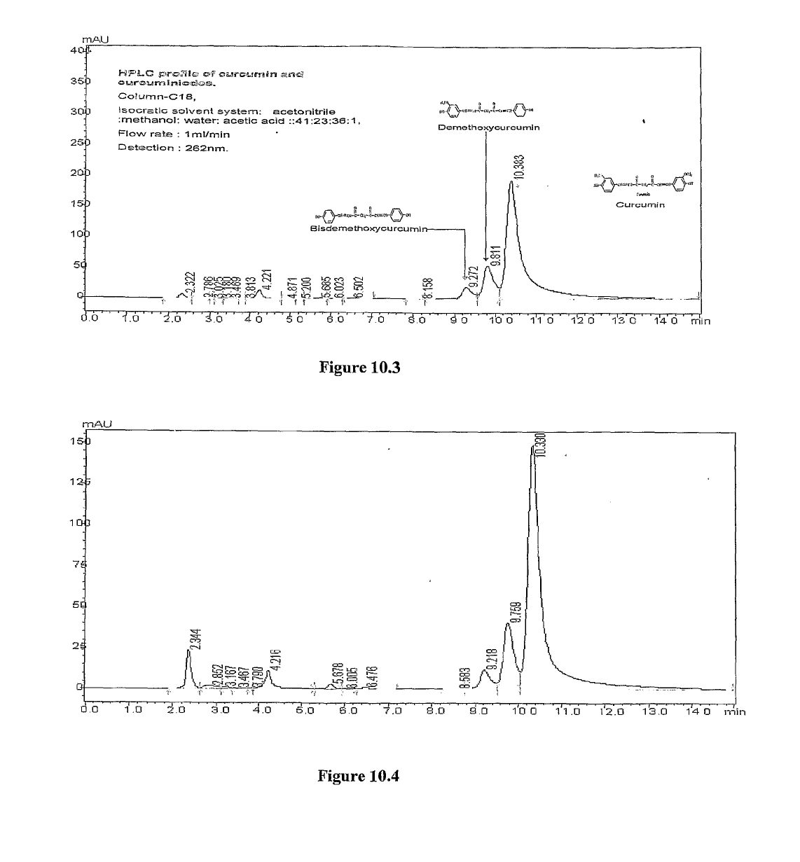

HPLC was performed using C18 column and isocratic solvent system consisting of acetonitrile: methanol: water: acetic acid::41:23:36:1, at a flow rate of 1 ml/min. Mass was determined by using MALDI-TOF mass spectrophotometer from Bruker Daltonik GmbH, (Germany). Curcumin was dissolved in ethanol while curcumin nanoparticles were resuspended in 20% ethanol and the mass spectra was recorded. Both curcumin and curcumin nanoparticles showed the presence of curcumin (mass 369), Demothoxy curcumin (339) and bisdemethoxy curcumin (309) indicating that the original molecules present in the curcumin sample are not modified by conversion to curcumin nanoparticles (FIGS. 10.1 and 10.2).

Viscosity of Nanoparticles: The viscosity of individual nanoparticle suspension was measured at room temperature and normal atmospheric pressure. The result indicates a change in viscosity of chitosan nanoparticles bound to curcumin from that of chitosan nanoparticles and curcumin nanoparticles (FIG. 1.4). This indicates binding of curcumin to chitosan which also correlates with changes in zetapotential of chitosan nanoparticles bound to curcumin from that of individual nanoparticles, indicating the binding of curcumin to chitosan.

TABLE 1
Summary of biophysical properties of the prepared nanoparticles

| | | Mean diameter of | | |
|---|---|---|---|---|
| | | nanoparticles | | |
| | Viscosity | (distribution of | | |
| | at | particle size ) | | |
| | 21.7° C. | measured by | | Zetapotential |
| Particles | in mPas | DLS | | (mV) |
| Chitosan | 5.64 | | | +331.2 |
| Solution(2% Cs in 1% | | | | |
| acetic acid) | | | | |
| Chitosan nanoparticles | 3.76 | 62.3 | (43.47-83.56) | +68.542 |
| loaded with curcumin | | | | |
| Curcumin nanoparticles | 1.53 | 115 | (50.02-283.21) | −131.372 |

### EXAMPLE 5

### Oral Bioavailability of Curcumin in Mice

Blood samples were obtained at different time intervals, that is, 30 min, 2 h, 4 h and 6 h after oral administration of curcumin (100 mg/kg through olive oil, 160 micrograms per mice through curcumin bound to Chitosan nanoparticles and 160 micrograms per mice through curcumin nanoparticles). Plasma was collected (after heparinization) by centrifugation at 4300 g for 10 min. Plasma (0.5 ml) was acidified to pH 3 using 6 N HCl and extracted twice (1 ml each) using a mixture of ethyl acetate and isopropanol (9:1; v/v,) by shaking for 6 min. The samples were centrifuged at 5000 g for 20 min. The organic layer was dried under inert conditions and the residue was dissolved in an eluent containing ethanol and filtered to remove insoluble material. The amount was quantitated from standard plot of curcumin in ethanol, by measuring the absorbance at 429 nm.

The identity of curcumin was established by HPLC (C18 column, isocratic solvent system acetonitrile: methanol: water: acetic acid::41:23:36:1, at a flow rate of 1 ml/min) and by MALD1-TOF mass spectrophotometer. (FIG. 10.1-10.4)

The increase in bioavailability of curcumin in terms of folds when compared to curcumin delivered through olive oil is depicted in FIG. 3.

The results show enhanced bioavailability of curcumin when fed through chitosan nanoparticles and as curcumin nanoparticles along with sustained release in the plasma till 6 hours.

TABLE 2.1
Extraction from plasma after 30 minutes post feeding

| | | Conc. of curcumin in micro | |
|---|---|---|---|
| | | grams extracted from | Percentage |
| Mice Group | Curcumin fed | 100 μl of plasma | Bioavailability |
| Curcumin in | 3 mg | 1.116 ± 0.146 | 0.036 ± 0.005 |
| olive oil | | | |
| Curcumin | 160 μg bound | 0.64 ± 0.072 | 0.396 ± 0.041 |
| bound to | to 200 μg of | | |
| chitosan | chitosan | | |
| nanoparticle | nanoparticle. | | |
| Curcumin | 160 μg | 0.836 ± 0.092 | 0.5 ± 0.060 |
| nanoparticle | | | |

TABLE 2.2
Extraction from plasma after 120 min

| | | Conc. of curcumin in | |
|---|---|---|---|
| | | micro grams extracted | Percentage |
| Mice Group | Curcumin fed | from 100 μl of plasma | Bioavailability |
| Curcumin in | 3 mg | 0.621 ± 0.037 | 0.020 ± 0.0006 |
| olive oil | | | |
| Curcumin | 160 μg bound to | 0.613 ± 0.020 | 0.376 ± 0.015 |
| bound on | 200 μg of | | |
| chitosan | chitosan | | |
| nanoparticle | nanoparticle. | | |
| Curcumin | 160 μg | 0.801 ± 0.059 | 0.496 ± 0.037 |
| nanoparticle | | | |

TABLE 2.3
Extraction from plasma after 240 min

| | | Conc. of curcumin in | |
|---|---|---|---|
| | | micro grams extracted | Percentage |
| Mice Group | Curcumin fed | from 100 μl of plasma | Bioavailability |
| Curcumin in | 3 mg | 0.366 ± 0.215 | 0.007 ± 0.001 |
| olive oil | | | |
| Curcumin | 160 μg bound to | 0.493 ± 0.080 | 0.306 ± 0.050 |
| bound on | 200 μg of | | |
| chitosan | chitosan | | |
| nanoparticle | nanoparticle. | | |
| Curcumin | 160 μg | 0.653 ± 0.094 | 0.403 ± 0.058 |
| nanoparticle | | | |

TABLE 2.4
Extraction from plasma after 360 min

| | | Conc. of curcumin in | |
|---|---|---|---|
| | | micro grams extracted | Bioavailability |
| Mice Group | Curcumin fed | from 100 μl of plasma | Percentage |
| Curcumin in | 3 mg | 0.079 ± 0.052 | 0.002 ± 0.001 |
| olive oil | | | |
| Curcumin | 160 μg bound to | 0.116 ± 0.020 | 0.072 ± 0.013 |
| bound on | 200 μg of | | |
| chitosan | chitosan | | |
| nanoparticle | nanoparticle. | | |
| Curcumin | 160 μg | 0.442 ± 0.584 | 0.046 ± 0.032 |
| nanoparticle | | | |

### EXAMPLE 6

### Antimalarial Activity of Curcumin Bound to Chitosan Nanoparticles/Curcumin Nanoparticles.

6.1 Experimental host and strain maintenance

Male Swiss mice weighing 25-30 g were maintained on a commercial pellet diet and housed under conditions approved by the Institutional Animal Ethics Commitee of the university. P. yeolli N-67 rodent malarial parasite, was used for infection. Mice were infected by intra peritoneal passage of 10⁶ infected erythrocytes diluted in phosphate buffered saline solution (PBS 10 mM, pH 7.4, 0.1 mL). Parasitemia was monitored by microscopic examination of Giemsa stained smears.

6.2 In Vivo Antimalarial Activity

In vivo antimalarial activity was examined in groups of 6 male Swiss mice (25-30 g) intraperitoneally infected on day 0 with P. yeolli such that all the control mice died between day 8 and day 10 post-infection. The mice were divided in to 4 groups of six mice each.

Untreated control group which was further subdivided into infected control group, olive oil control group and chitosan control group
1. Group treated with curcumin in olive oil control group
2. Group treated with curcumin on chitosan nanoparticles
3. Group treated with curcumin nanoparticles

For the group treated with curcumin in olive oil, curcumin was suspended in olive oil (100 mg/kg body weight). They were given curcumin at a dose of 3 mg/mice once, suspended in olive oil through the oral route. For the group treated with curcumin bound to chitosan nanoparticles and curcumin nanoparticles, 160 micrograms of curcumin (through chitosan or curcumin nanoparticles) was made available per mouse and was introduced by means of feeding gauge into the oral cavity of non-anesthetized mice as daily doses.

Each of the groups was infected with 1×10⁶ red blood cells taken from an animal having approximately 30% parasitemia. Treatment, in each case, was started only when individual mouse showed parasitemia of 1-3%, that is, by the 4^{th} day of infection. Survival of mice was monitored for a period of 120 days.

All the mice in the infected control group and olive oil control group died between 7^{th} to 11^{th} day post-infection (FIG. 4.1-4.2). All the mice in the chitosan control group died between 7^{th} to 12^{th} day post infection (a delay of two days in comparison to the infected control and olive oil control groups) (FIG. 4.3).

In the group treated with curcumin in olive oil control, 2 out of the 6 mice survived for more than 100 days after cure while 4 died between 10^{th} to 12^{th} day post infection (FIG. 4.4).

All the mice survived in the groups treated with curcumin bound to chitosan nanoparticles and curcumin nanoparticles. All of the mice survived for more than 100 days after cure and were resistant to reinfection by the same parasite (FIG. 4.5-4.6).

### EXAMPLE 7

### Intracellular Localization of Curcumin in Infected Erythrocytes

7.1 Intracellular Accumulation of Curcumin in Infected RBC

Infected Mice with different parasitemia (0% to 17.8%) were given curcumin bound to chitosan nano particles orally. Red blood cells were purified from each mice by density gradient centrifugation and curcumin fluorescence was detected by using FACS. FACS data showing curcumin fluorescence intensity of uninfected and infected RBCs is depicted in FIG. 5.2-5.3.

7.2 Quantitative Estimation of Curcumin Localized/Accumulated in Erythrocytes (Both Infected/Normal)

Red blood cells from both control and infected mice were purified by density gradient centrifugation, and curcumin was extracted out from 1×10⁸ red blood cells using the procedure as described in example 5 and the result shows more accumulation of curcumin in RBC having higher level of parasitemia as indicate in the FIG. 5.5.

7.3 Accumulation of Curcumin in Infected Red Blood Cells by Confocal Microscopy

Slides for confocal microscopy were prepared by fixing erythrocytes or lymphocytes separated by density gradient centrifugation using ficoll from non infected Plasmodium yoelli infected mice fed with curcumin nanoparticles. The isolated cells (erythrocytes) were then sealed with cover slip using mounting medium. Fluorescence imaging of cells was performed with an Olympus Fluoview 500 confocal laser-scanning microscope (Olympus, Tokyo, Japan) equipped with a multi-Argon laser for excitation at 458, 488 and 515 nm. The images were acquired either with 20× objective or a 60× water immersion objective using the fluoview software (Olympus, Tokyo, Japan). The curcumin emission was collected using the barrier filter BA505. The excitation wave length was 458 nm for curcumin. FIG. 5.6-5.7.

### EXAMPLE 8

### In Vivo Inhibition of Hemozoin Synthesis by Chloroquinine as Well as Curcumin

Infected mice were divided into 4 groups (each having 4 mice), namely:
1. Control group which was further sub-divided into the infected control group, olive oil control group and chitosan control group
2. Infected and fed with Chloroquinine (1.7 mg in 100 μl of normal saline/mouse/day orally)
3. Infected and fed with Curcumin bound to chitosan nanoparticles (160 μg of curcumin bound to 200 μg of chitosan nanoparticles/per mouse/twice a day) through oral route
4. Infected and fed with Chitosan nanoparticles (200 micrograms of chitosan/day) orally

Treatment in each group except the control was started when parasitemia had reached ˜10% in each mouse and was carried out for 3 days. Red blood cells were purified on the third day of treatment. Approximately 4×10⁷ cells were suspended in 25 mM Tris HCl pH 7.8 containing 2.5% SDS. The cells were centrifuged at 10,000 g for 10 min, supernatant was discarded and the pellet washed in 1 ml of 0.1 M alkaline bicarbonate buffer (pH 9.2). The washed pellet was dissolved in 0.05 ml of 2 N sodium hydroxide and absorbance was read at 400 nm after dilution to 1 ml using 2.5% SDS solution in water. The concentration of heme was calculated by using 90.8 as the milli Molar Extinction coefficient of heme.

The results of in vivo inhibition of hemozoin synthesis in P. yoelii infected mice by feeding chloroquinine in normal saline or curcumin bound to chitosan nanoparticles (hemozoin concentration is measured in terms of dissociated heme) is depicted in FIG. 6.

### EXAMPLE 9

### Detection of Apoptosis

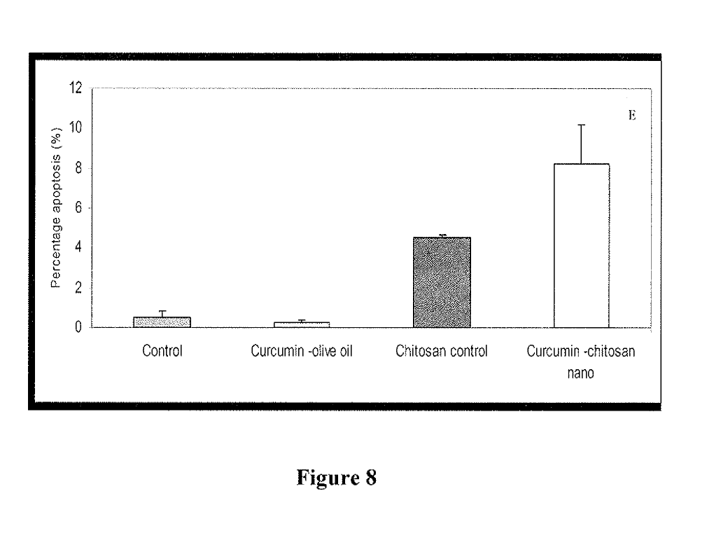

Terminaldeoxynucleotidyl transferase-mediated deoxyuridine triphosphate biotin nick-end labelling (TUNEL) was performed using the ApoAlert™ DNA Fragmentation Assay kit (R&D Systems). Parasitic cells were isolated from infected RBCs from different groups by density gradient centrifugation. The parasitic cells were washed twice with 1 ml PBS and fixed with 4% formaldehyde/PBS for 25 min at 4° C. After two washes with PBS, the pellet was resuspended in 5 ml permeabilization solution (0.2% Triton X-100 in PBS) and incubated on ice for 5 minutes. Eighty microlitres of equilibration buffer was added and was incubated at room temperature for 5 minutes. The cells were labeled by adding 50 ml TUNEL mix followed by incubation for 60 minutes at 37° C. in a dark, humidified incubator. One millilitre of 20 mM EDTA was then added to terminate the tailing reaction. The samples were washed with PBS and the pellet was resuspended in 250 ml PBS for flow cytometry analysis. The results of this experiment are depicted in FIGS. 7 and 8.

### EXAMPLE 10

### Toxicological Studies

Toxicological studies were carried out on five groups of swiss albino mice and five groups of male wister rats as per the details in table 3.

TABLE 3
Toxicological Study using mice and rats fed with PBS, Curcumin
in Olive oil, Chitosan nano particles bound to curcumin,
Chitosan nano particles and Curcumin nanoparticles

| Group | Mice | Rat |
|---|---|---|
| Group-1 | 6 female swiss albino mouse. | 6 male wister rats |
| PBS | Given 100 microliters of | Given 1 ml of PBS |
| | PBS orally for 14 days. | orally for 14 days. |
| Group-2 | 6 female swiss albino mouse. | 6 male wister rats |
| Curcumin in | Given 4 mg of curcumin | Given 40 mg of curcumin |
| olive oil | suspended in 100 microliters | suspended in 1 ml of olive |
| | of olive oil orally for 14 days. | oil orally for 14 days. |
| Group-3 | 6 female swiss albino mouse. | 6 male wister rats |
| Chitosan | Given 4 mg of curcumin | Given 40 mg of curcumin |
| nano | bounded to 4 mg of chitosan | bounded to 40 mg of |
| bounded to | nanoparticles orally for 14 | chitosan nano particles |
| curcumin | days | orally for 14 days |
| Group-4 | 6 female swiss albino mouse. | 6 male wister rats |
| Chitosan | Given 4 mg of chitosan | Given 40 mg of chitosan |
| nano | nanoparticles suspended in | nanoparticles suspended |
| | 100 microliters of PBS | in 1 ml of PBS |
| | orally for 14 days | orally for 14 days |
| Group-5 | 6 female swiss albino mouse. | 6 male wister rats |
| Curcumin | Given 4 mg of curcumin | Given 40 mg of curcumin |
| nanoparticle | nanoparticles suspended in | nanoparticles |
| | 100 microliters of PBS | suspended in 1 ml |
| | orally for 14 days | of PBS orally for 14 days |

### EXAMPLE 10a

### Histopathological Examination

Histopathological examination of organs was completed in six animals from each group. The organ taken for histological study from each animal included brain, liver, kidney and heart. Eosin and hematoxylin stained section were available for study from all these organs. No histological evidence of damage to the liver, heart, brain or kidney was seen in any animal in any group. The histological features clearly indicate that the preparations administered by the oral route, that is, curcumin in olive oil, curcumin bound to chitosan nanoparticles, chitosan nanoparticles and curcumin nanoparticles are non-toxic in Wister Rats and Swiss Albino mice.

### EXAMPLE 10b

### Biochemical Analysis of Mouse and Rat Blood Samples

Blood samples from members of the five groups of Swiss Albino Mice and Wister Rats after oral feeding to PBS, curcumin in olive oil, curcumin bound to chitosan nanoparticles, chitosan nanoparticles and curcumin nanoparticles as directed in table 3, were subjected to determination of serum glutamic oxaloacetic transaminase (SCOT) level, serum glutamic pyruvic transaminase (SGPT) level, serum urea level, serum creatinine level, serum cholesterol level, serum albumin level and serum hemoglobin level.

No rise was seen in the serum SGOT, SGPT, urea and creatinine levels after oral feeding of PBS, curcumin in olive oil, curcumin bound to chitosan nanoparticles, chitosan nanoparticles and curcumin nanoparticles. The serum levels of cholesterol, albumin and hemoglobin were also not significantly altered. This indicates that the curcumin nanoparticles of the present invention are non-toxic and safe.

### EXAMPLE 11

### Effect on Fasting Blood Sugar Levels in Human Volunteers

Curcumin nanoparticles at a dose of 500 mg/day/person were given orally to nine human volunteers (1, 3, 4, 6, 8, 9, 10, 11 & 12) who gave their informed consent to participate in the study. Their blood glucose level was measured under fasting conditions before the start of the experiment (dark spots) and after 15 day of continuous oral consumption of same quantity of curcumin nanoparticles (white spots) Normal curcumin was given orally to another group of seven human volunteers (2, 5, 7, 13, 14, 15 & 16) at a dose of 500 mg/day/person. The results of the analysis are depicted in FIG. 11. While fasting glucose level was not altered in the curcumin control group there was a significant decrease in the Nanocurcumin group indicating its ability to lower blood glucose level.

### EXAMPLE 12

### Effect on Kidney Function in Human Volunteers

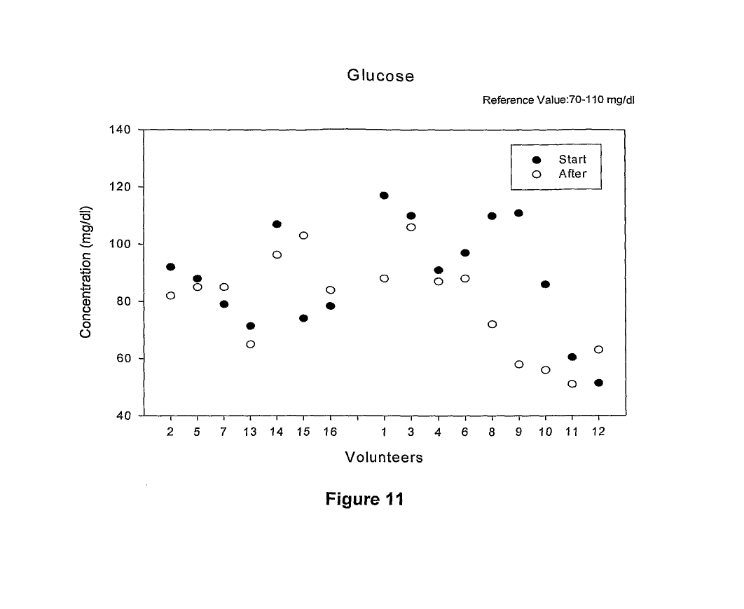

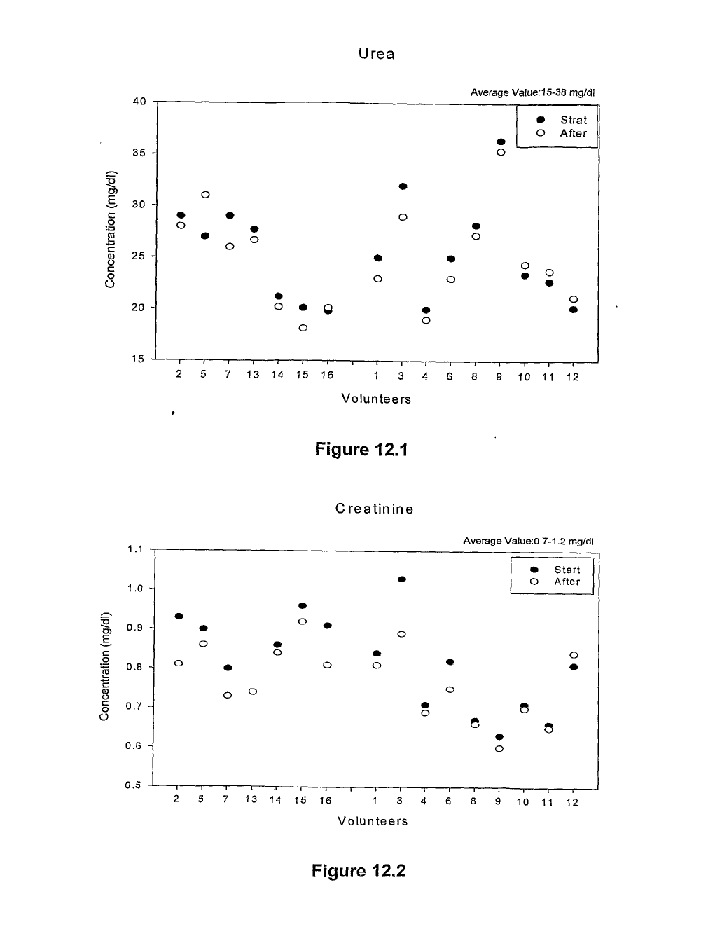

Curcumin nanoparticles at a dose of 500 mg/day/person were given orally to nine human volunteers (1, 3, 4, 6, 8, 9, 10, 11 & 12) who gave their informed consent to participate in the study. Normal curcumin was given orally to another group of seven human volunteers (2, 5, 7, 13, 14, 15 & 16) at a dose of 500 mg/day/person. The level of serum urea, creatinine and potassium (In case of potassium human volunteers(1, 3, 4, 6 were given curcumin nanoparticles where as 2, 5, 7 were given normal curcumin) were measured before the start of the experiment (dark spots) and after 15 day of continous oral comsumption of same quantity of curcumin nanoparticles (white spots). Results of said tests are depicted in FIGS. 12.1- 12.3. The serum creatinine, urea and potassium levels (7 Volunteers) of all the volunteer under the study were within the normal range both before and after 15 days of continous oral consumption. There is slight decrease in serum creatinine and urea levels and increase in potassium level indicating tubular reabsorption of potassium by kidney, thereby showing an overall beneficial effect of curcumin on kidney.

### EXAMPLE 13

### Effect on Cardiovascular function in Human Volunteers

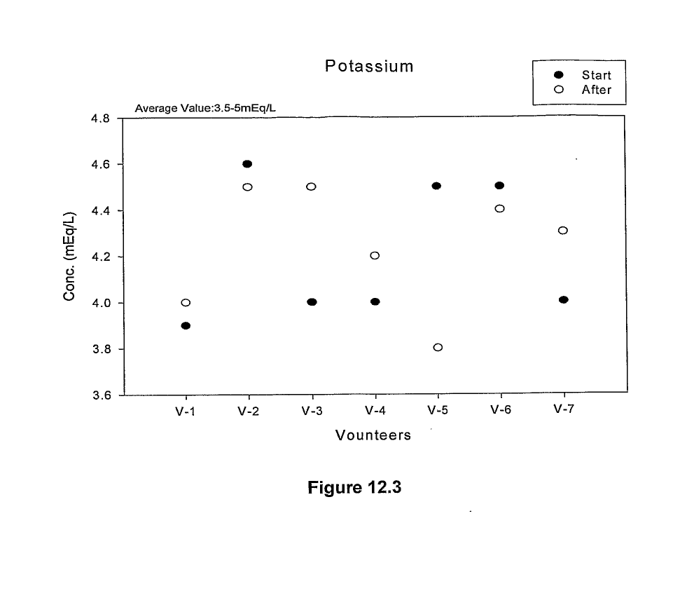

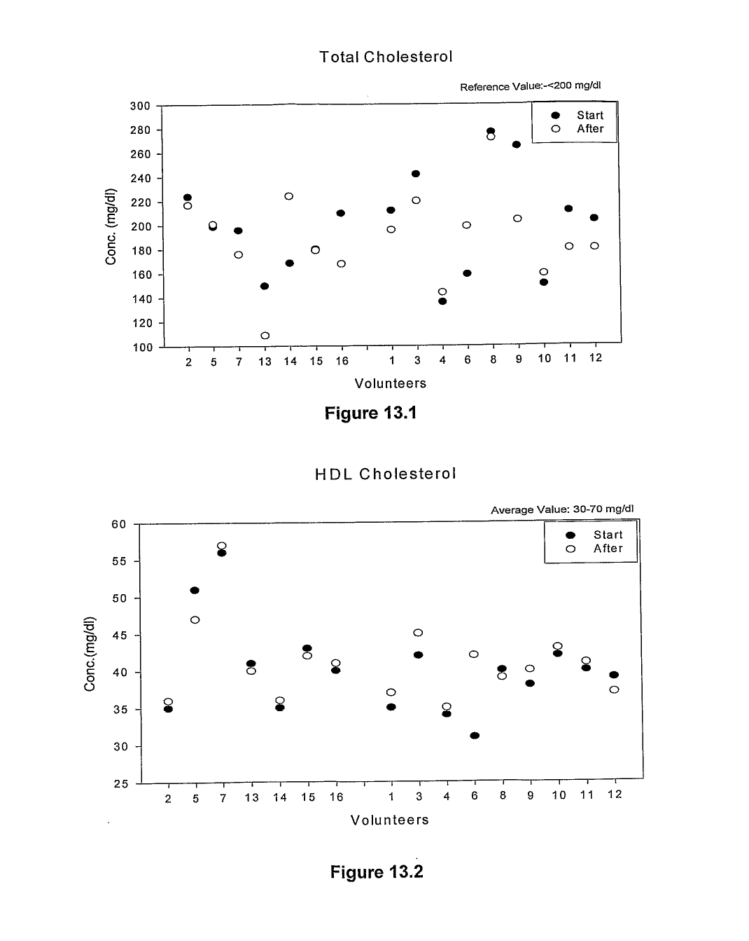

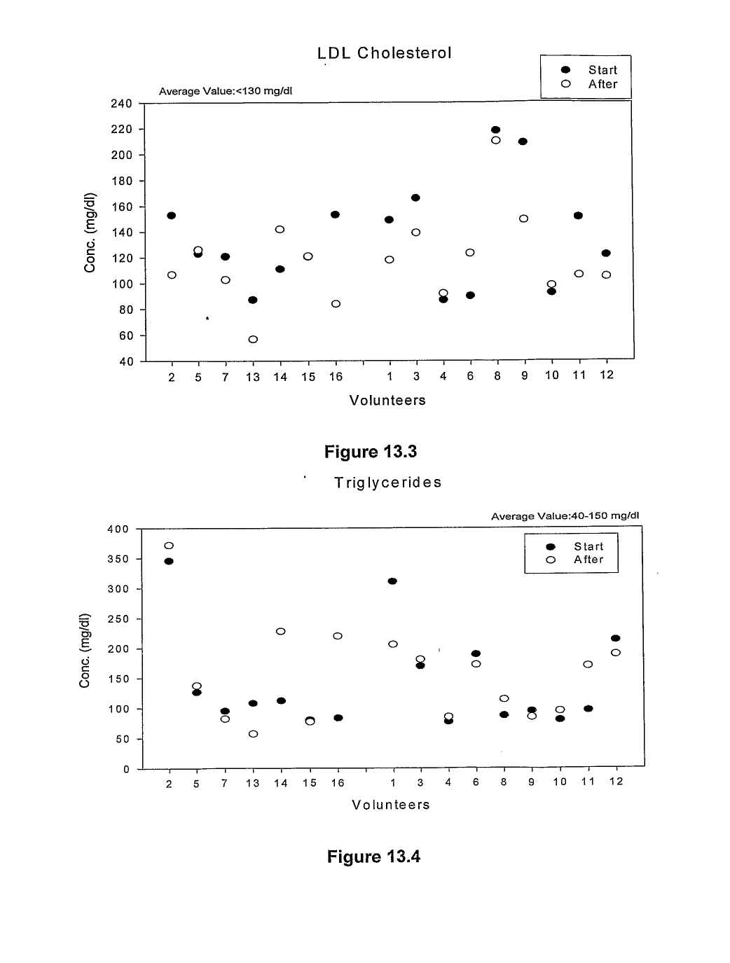

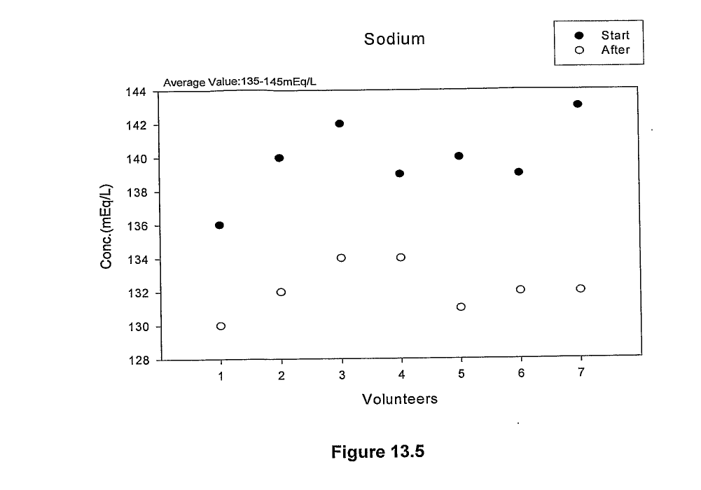

Curcumin nanoparticles at a dose of 500 mg/day/person were given orally to nine human volunteers(1, 3, 4, 6, 8, 9, 10, 11 & 12) who gave their informed consent to participate in the study. Normal curcumin was given orally to another group of seven human volunteers (2, 5, 7, 13, 14, 15 & 16) at a dose of 500 mg/day/person. The level were measured before the start of the experiment (dark spots) and after 15 day of continous oral comsumption of same quantity of curcumin nanoparticles (white spots). The effect of curcumin and nanocurcumin was studied on the levels of serum total cholesterol, HDL cholesterol, LDL cholesterol, triglycerides and sodium (In case of sodium only seven human volunteers 1, 3, 4, 6 were given curcumin nanoparticles where as 2, 5, 7 were given normal curcumin). Results of said tests are depicted in FIGS. 13.1-13.5. A decline in total cholesterol level was seen in the nanocurcumin group consistently as compared to normal curcumin group. Furthermore there is a marked increase in HDL cholesterol (good cholesterol) in case of curcumin nanoparticle group. Level of LDL cholesterol (bad cholesterol) and triglycerides were lowered consistently in curcumin nanoparticle group as compared to normal curcumin group. Decrease in serum sodium level was also observed indicating the promising anti-cholesterolic, anti-stroke, and other beneficial effects on cardiovascular diseases.

### EXAMPLE 14

### Effect of Oral Intake of Curcumin and Nanocurcumin on Hemoglobin and Rbc Level of Human Volunteers

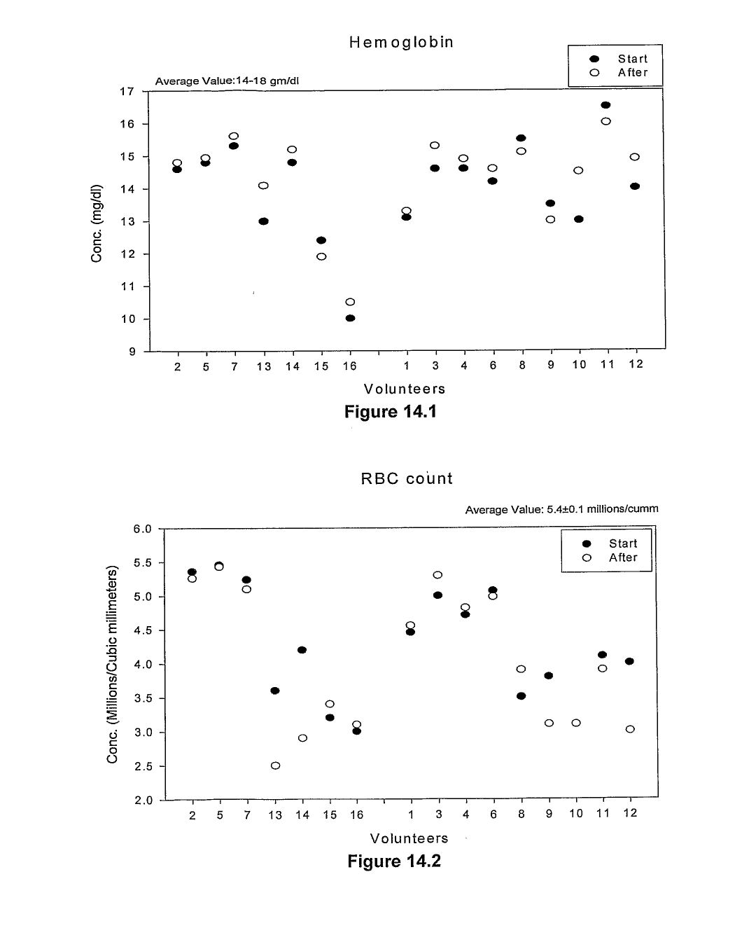

Curcumin nanoparticles at a dose of 500 mg/day/person were given orally to nine human volunteers (1, 3, 4, 6, 8, 9, 10, 11 & 12) who gave their informed consent to participate in the study. Normal curcumin was given orally to another group of seven human volunteers (2, 5, 7, 13, 14, 15 & 16) at a dose of 500 mg/day/person. The levels were measured before the start of the experiment (dark spots) and after 15 day of continuous oral consumption of same quantity of curcumin nanoparticles (white spots) The effect of curcumin and nanocurcumin was studied on the levels of blood hemoglobin and RBCs. Results of said tests are depicted in FIGS. 14.1-14.2, which indicates that there is no adverse effect in terms of induction on anemic condition or lowering of RBC counts following the treatment regime.( ).

### EXAMPLE 15

### Effect on Liver Inflammation in Human Volunteers

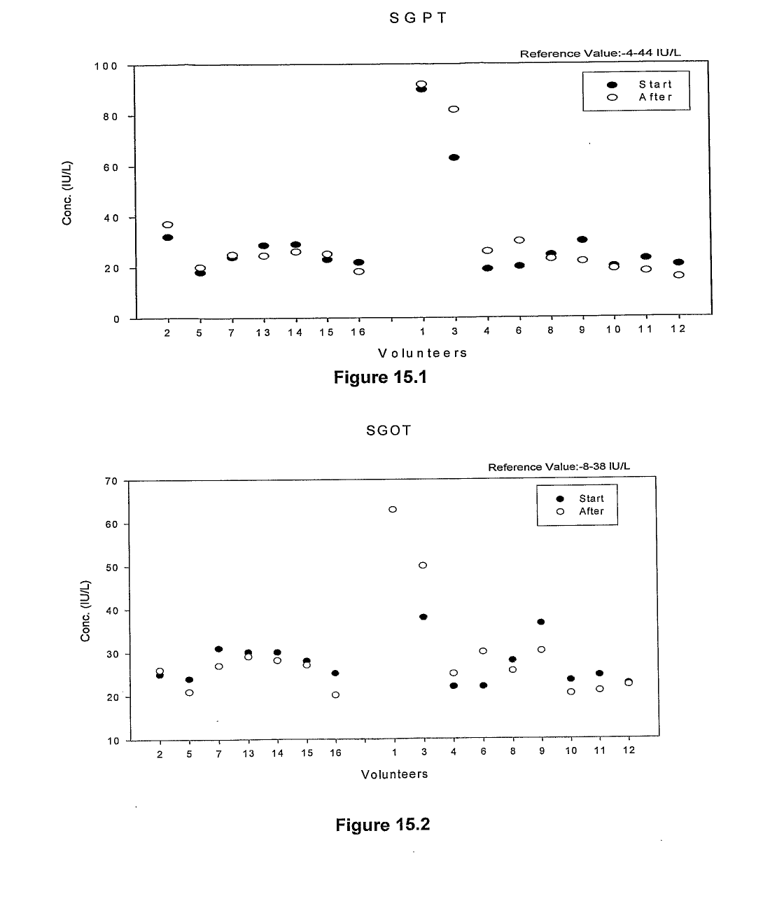

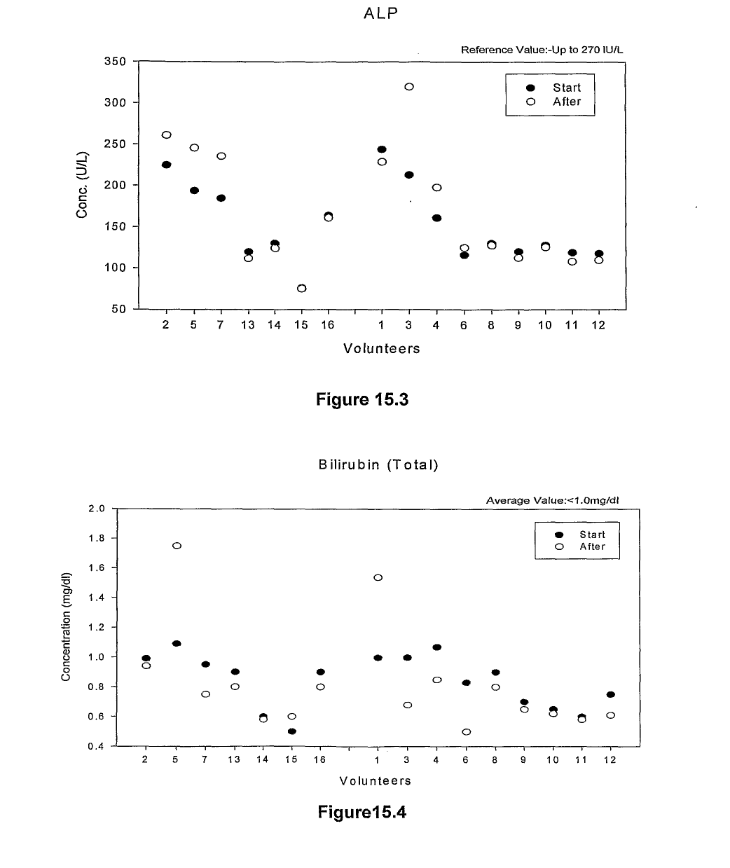

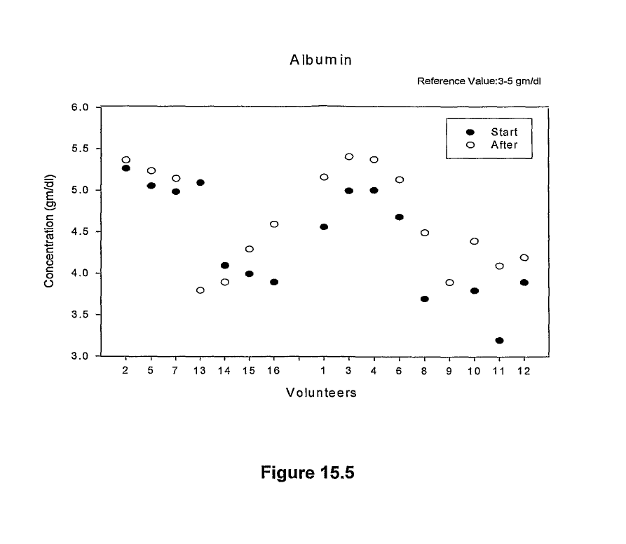

Curcumin nanoparticles at a dose of 500 mg/day/person were given orally to nine human volunteers (1, 3, 4, 6, 8, 9, 10, 11 & 12) who gave their informed consent to participate in the study. Normal curcumin was given orally to another group of seven human volunteers (2, 5, 7, 13, 14, 15 & 16) at a dose of 500 mg/day/person. The level were measured before the start of the experiment (dark spots) and after 15 day of continuous oral consumption of same quantity of curcumin nanoparticles (white spots). The effect of curcumin and nanocurcumin was studied on the levels of serum SGPT, SGOT, ALP, albumin and bilirubin. Results of said tests are depicted in FIGS. 15.1-15.5. It is apparent that SGOT and SGPT levels are not significantly altered and albumin levels are increased in naocurcumin treated group indicating that nanocurcumin is good for the liver. The ALP and Bilirubin levels were also in the normal range except in one or two cases showing that curcumin and nanocurcumin do not have any adverse effect on liver function.

### EXAMPLE 16

### Effect of Oral Intake of Curcumin and Nanocurcumin on Globulin Level, Eosinophils and Neutrophils Count and Platelet Count of Human Volunteers

Curcumin nanoparticles at a dose of 500 mg/day/person were given orally to nine human volunteers (1, 3, 4, 6, 8, 9, 10, 11 & 12) who gave their informed consent to participate in the study. Normal curcumin was given orally to another group of seven human volunteers (2, 5, 7, 13, 14, 15 & 16) at a dose of 500 mg/day/person. The level were measured before the start of the experiment (dark spots) and after 15 day of continuous oral consumption of same quantity of curcumin nanoparticles (white spots).

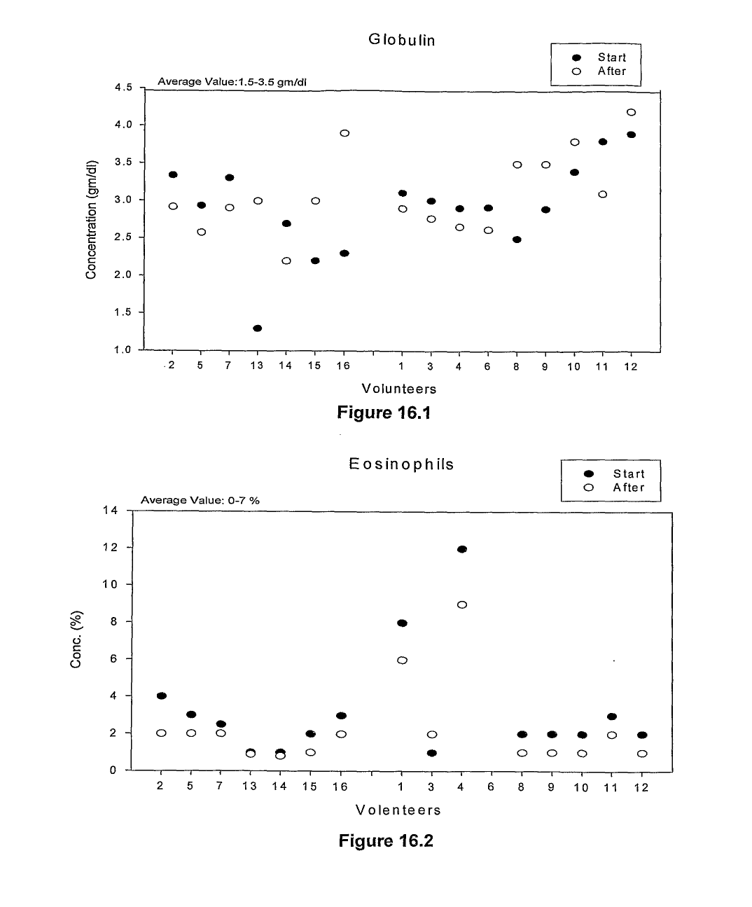

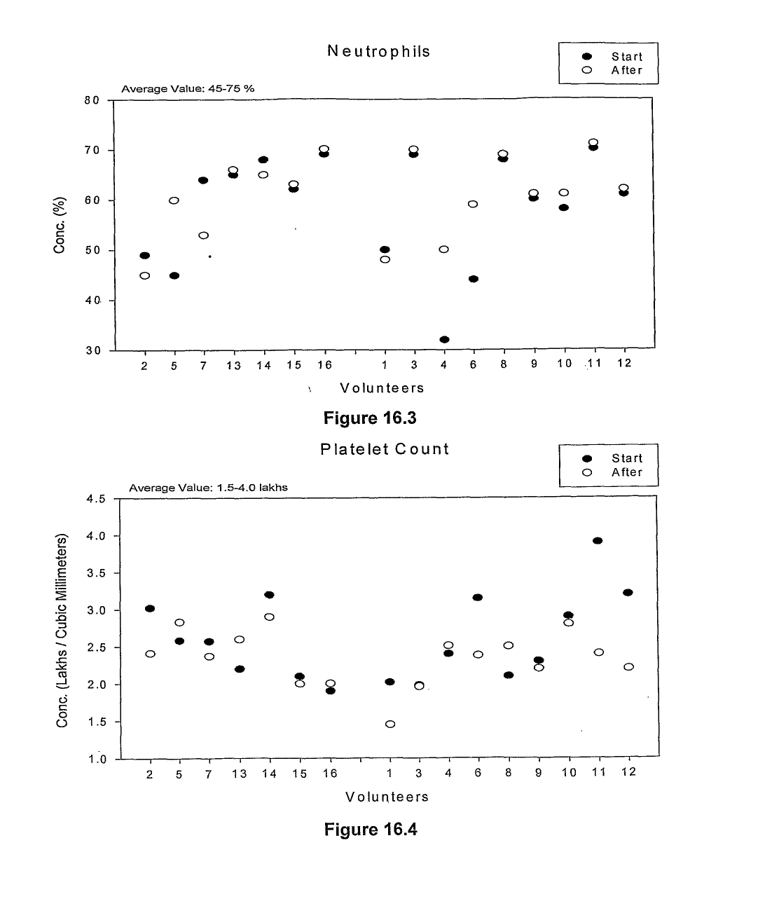

Results of said tests are depicted in FIGS. 16.1-16.4. The result indicates that there is no significant effect of curcumin on the levels of eosinophiles, neutrophils and platles.

### EXAMPLE 17

### Anti-Malaria Effect of Nanocurcumin

Patients suffering from malaria were administered nanocurcumin capsules after having their informed consent under the supervision of a traditional medicine practitioner at a dose of 200 mg twice daily for 5 to 7 days for Plasmodium vivax cases and 200 mg four times per day for 5 to 7 days for Plasmodium falciparum cases. All nine patients were cured (table 4). Another group of five patients were studied for relapse. The patients who were cured did not show any relapse for at least 9 months. (table 5).

TABLE 4
Details of Malaria Treatment with Nanocurcumin

| | | | | | Examined | |
|---|---|---|---|---|---|---|
| Serial | | | | Start of | for parasite | Remarks/ |
| no | Age | sex | Diagnosis | Treatment | in the blood | relaps |
| 1 | 11 | F | Infected | 15 Jul. 2009 | 20 Jul. 2009 | Cured |
| | | | with both | | no parasite | |
| | | | Plasmodium | | or parasite | |
| | | | vivax and | | antigen | |
| | | | Plasmodium | | detected | |
| | | | falciparum | | | |
| 2 | 45 | M | Infected with | 16 Jul. 2009 | 21 Jul. 2009 | Cured |
| | | | P. falciparum | | no parasite | |
| | | | | | or parasite | |
| | | | | | antigen | |
| | | | | | detected | |
| 3 | 29 | M | Infected | 10 Jul. 2009 | 15 Jul. 2009 | Cured |
| | | | with both | | no parasite | |
| | | | P. vivax and | | or parasite | |
| | | | P. falciparum | | antigen | |
| | | | | | detected | |
| 4 | 8 | M | Infected with | 10 Jul. 2009 | 15 Jul. 2009 | Cured |
| | | | P. falciparum | | no parasite | |
| | | | | | or parasite | |
| | | | | | antigen | |
| | | | | | detected | |
| 5 | 23 | F | Infected with | 12 Jul. 2009 | 17 Jul. 2009 | Cured |
| | | | P. falciparum | | no parasite | |
| | | | | | or parasite | |
| | | | | | antigen | |
| | | | | | detected | |
| 6 | 4 | M | Infected with | 13 Aug. 2009 | 21 Aug. 2009 | Cured |
| | | | P. vivax | | no parasite | |
| | | | | | or parasite | |
| | | | | | antigen | |
| | | | | | detected | |
| 7 | 12 | M | Infected with | 28 Aug. 2009 | 12 Sep. 2008 | Cured |
| | | | P. vivax | | no parasite | |
| | | | | | or parasite | |
| | | | | | antigen | |
| | | | | | detected | |
| 8 | 5 | M | Infected with | 1 Aug. 2009 | 12 Sep. 2008 | Cured |
| | | | P. vivax | | no parasite | |
| | | | | | or parasite | |
| | | | | | antigen | |
| | | | | | detected | |
| 9 | 19 | M | Infected with | 2 Sep. 2008 | 11 Sep. 2008 | Cured |
| | | | P, vivax | | no parasite | |
| | | | | | or parasite | |
| | | | | | antigen | |
| | | | | | detected | |

TABLE 5
Details of Malaria Treatment and Realapse Studies in
patients treated with Nanocurcumin

| | | | | | Examined | |
|---|---|---|---|---|---|---|
| | | | | | for | |
| Serial | | | | Start of | parasite in | Remarks/ |
| no | Age | sex | Diagnosis | Treatment | the blood | relaps |
| 1 | 42 | M | Infected with | 4 Jul. 2008 | 12 Jul. 2008 | No relapse |
| | | | Plasmodium | | | reported |
| | | | vivax | | | since 1 year |
| | | | | | | after cure |
| 2 | 37 | F | Infected with | 9 Aug. 2008 | 30 Aug. 2008 | No relapse |
| | | | Plasmodium | | | reported |
| | | | vivax | | | since |
| | | | | | | 11 months |
| | | | | | | after cure |
| 3 | 33 | M | Infected with | 8 Sep. 2008 | 20 Sep. 2008 | No report |
| | | | Plasmodium | | | of relapse |
| | | | vivax | | | since |
| | | | | | | 10 months |
| | | | | | | after cure |
| 4 | 19 | M | Infected with | 10 Sep. 2008 | 20 Sep. 2008 | No report |
| | | | Plasmodium | | | of relapse |
| | | | vivax | | | since |
| | | | | | | 10 months |
| | | | | | | of cure |
| 5 | 45 | M | Infected with | 10 Oct. 2008 | 25 Oct. 2008 | No report |
| | | | Plasmodium | | | of relapse |
| | | | vivax | | | since |
| | | | | | | 9 months |
| | | | | | | after cure. |

1. Nano-sized particles of pure curcumin wherein said nano-sized particles comprise about 100% curcumin.

2-11. (canceled)

12. The nano-sized particles of pure curcumin as claimed in claim 1, wherein the diameter of said particles ranges between 50 nm to 284 nm.

13. The nano-sized particles of pure curcumin as claimed in claim 1, wherein the mean diameter of said particles is 115 nm.

14. Nanoparticles comprising curcumin bound to chitosan nanoparticles, wherein said chitosan nanoparticles comprise chemically unmodified chitosan.

15. The nanoparticles as claimed in claim 14 comprising curcumin coated on the surface of chitosan nanoparticles.

16. The nanoparticles as claimed in claim 14, wherein the diameter of the nanoparticles ranges between 43 nm to 84 nm.

17. The nanoparticles as claimed in claim 14, wherein the mean diameter of the nanoparticles is 62.3 nm.

18. A process of preparing nano-sized particles of pure curcumin comprising:
dissolving curcumin in alcohol to obtain a solution comprising curcumin and alcohol; and
spraying said solution comprising curcumin and alcohol at 25 ° C.-40° C. under a nitrogen atmosphere and high pressure into a second solution comprising a low percentage of an organic acid while stirring at room temperature.

19. A process of preparing nanoparticles comprising curcumin bound to chitosan nanoparticles comprising:
(a) making a clear solution of chitosan in an organic acid by stirring the suspension while heating at 50° C.-80 ° C.;
(b) rapidly cooling the solution of (a) to 4° C.-10° C. and repeating steps (a) and (b);
(c) heating the clear solution at 50° C.-80° C. and spraying said clear solution under pressure into water while stirring at 4° C-10° C. to obtain chitosan nanoparticles;
(d) preparing a clear solution of curcumin in alcohol and adding it to a stirred aqueous suspension of chitosan nanoparticles in an organic acid and stirring the resulting suspension at room temperature; and
(e) centrifuging the curcumin-chitosan nanoparticles suspension and repeating the process to remove unbound curcumin from the nanoparticles.

20. A method of using the curcumin nanoparticles of claim 1 to treat a disease or disorder selected from the group consisting of: cancers, inflammatory diseases, alzeihmer's disease, cholesterol gall stone, diabetes, alcohol and drug induced liver diseases, microbial infections, parasitic infestation, malaria and other parasitic diseases, and neurological disorders comprising providing the curcumin nanoparticles of claim 1 to a subject in need thereof

21. A medicament comprising the curcumin nanoparticles of claim 1.
